(19) United States
(12) Patent Application Publication
(10) Pub. No.: US 2025/0101403 A1
(43) Pub. Date: Mar. 27, 2025

(54) INTEGRASES

(71) Applicant: Seamless Therapeutics GmbH, Dresden (DE)

(72) Inventors: Aksana SCHNEIDER, Dresden (DE); Felix Johannes LANSING, Nurnberg (DE)

(21) Appl. No.: 18/822,341

(22) Filed: Sep. 2, 2024

(30) Foreign Application Priority Data
Sep. 1, 2023 (EP) 23194978.5
Feb. 7, 2024 (EP) 24156399.8

(51) Int. Cl. C12N 9/00; A61K 38/00; C12N 15/90; C40B 30/08

(52) U.S. Cl. CPC C12N 9/93; C12N 15/902; C40B 30/08; A61K 38/00; C12Y 605/01

(57) ABSTRACT

The present invention pertains to a method for generating a target-specific large serine recombinase (LSR), the method comprising the steps of: a) generating a plurality of first variants of an LSR by introducing one or more mutations into a sequence encoding the LSR; b) generating a library of expression vectors, wherein each expression vector comprises a first region encoding one of the first variants of an LSR, and a second region comprising at least two first target sites of the LSR, wherein the two first target sites are different from each other; c) introducing the library of expression vectors into host cells; d) culturing the host cells and expressing the first variant LSR; e) isolating plasmid DNA from the culture of host cells; f) determining whether a portion of the second region of the expression vector between the two first target sites has been excised by the variant LSR; g) generating a plurality of second LSR variants by introducing one or more mutations into the sequence encoding those first LSR variants that excised the portion of the second region of the expression vector between the two first target sites; and h) repeating steps b) to f) optionally with at least one second target site, wherein the at least one second target site differs from the first target sites in at least one nucleotide. The present invention further provides variant LSRs obtained by the method, a nucleic acid or group of nucleic acids encoding a variant LSR, a vector comprising said nucleic acid(s); a system for integrating a donor DNA into a target nucleic acid; and uses of the LSRs, the nucleic acid(s) or the vector.

### RELATED APPLICATIONS

This application claims priority to European Patent Application Nos. 24156399.8, filed Feb. 7, 2024, and 23194978.5, filed Sep. 1, 2023, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Aug. 28, 2024, is named 757657_TUD9-004_ST26.xml and is 17,587,618 bytes in size.

The present invention relates to a method for generating target-specific large serine recombinases, also called integrases. The present invention further pertains to large serine recombinase variants obtained by said method and systems comprising the same, nucleic acids encoding the same, vectors expressing the nucleic acids, as well as uses of said large seine recombinases for integrating a donor nucleic acid sequence into a target nucleic acid sequence, preferably into the genome of a subject or cell.

### BACKGROUND OF THE INVENTION

Genome integration, the process of introducing foreign DNA into the genome of an organism, is a fundamental technique in molecular biology and genetic engineering. In the realms of medicine and biotechnology, the ability to precisely manipulate genomes is of paramount importance for advancements in therapeutics, disease research, and biopharmaceutical production.

One crucial aspect of genome editing is the site-specific integration of genetic payloads into desired genomic loci. Integrating sizable genetic payloads at precise genomic locations offers numerous advantages, such as targeted gene therapy, precise gene regulation, and the creation of genetically modified organisms for various research and industrial purposes. Targeted integration allows the user to insert the transgene into a locus that favors stable, long-term expression. It helps to avoid strong positional effects and reduce unwanted effects on cell functions, as well as to control the copy numbers at each integration event. However, achieving target site-specific integration of large DNA fragments remains a significant challenge in current genome editing methodologies, currently requiring inefficient, multi-step processes that are time and resource intensive (Anzalone et al. 2022; Yarnall et al. 2023; Zhu et al. 2014).

Large serine recombinases (LSR), also known as integrases, have emerged as highly valuable tools for facilitating precise and efficient genome integration. They are a family of enzymes encoded in temperate phage genomes or on mobile elements, which precisely cut and recombine DNA in a highly controllable and predictable way. In phage integration, LSRs act at specific sites, i.e. at the attP site in the phage and the attB site in the host chromosome, where cleavage and strand exchange lead to the integrated prophage flanked by the recombinant sites attL and attR. To perform integration, LSRs do not require any additional factors, auxiliary DNA sequences, or specific substrate topologies (Smith, 2015). The first LSRs to be studied in in vitro recombination systems were the integrases from the Streptomyces phage PhiC31 (Thorpe and Smith, 1998) and Mycobacteriophage Bxb1 (Kim et al., 2003).

Integrases typically consist of a relatively conserved catalytic N-terminal domain (1-140 aa) required for DNA cleavage and for rejoining, a “recombinase” domain, and a zinc ribbon domain. The catalytic and “recombinase” domains are linked by the long aE helix and the “recombinase” domain and the zinc ribbon domain are connected by a short linker. The “recombinase” domain and the zinc ribbon domain are collectively termed the C-terminal domain and range in size from 319 to about 550 amino acids. The zinc ribbon domain contains a long flexible coiled-coil motif implicated in additional binding specificity and irreversibility of forward integration reaction without excision cofactors (reviewed in Van Duyne and Rutherford, 2013; Smith, 2015).

The attachment sites used by LSRs comprise the binding sites for their cognate integrase; no host or accessory factors bind to the attachment sites. The attP and attB sites show a relatively simple structure, typically having less than 50 bp, with the crossover site centrally located and flanked by imperfect inverted repeats. Sites forming after recombination are called attL and attR, and contain half sites from attP and attB. Recombination by LSRs is strongly directional. Only attB and attP sites will undergo recombination; the resulting attL and attR sites are inactive in the absence of a phage-encoded factor required for excision, and no other pairing of sites (e.g., attB and attB) will lead to recombinant products (Ghosh et al., 2005; Rutherford et al., 2013; Thorpe et al., 2000; Smith, 2015).

The following mechanism of recombination has been proposed for the LSR family: Integrases bind to their target attachment sites as dimers and then form a tetramer, bringing the DNA sites together in a synaptic complex. Within this complex, all four subunits are simultaneously activated, and the catalytic serine residues break all four DNA strands simultaneously. Each cleaved end remains covalently bound to an integrase monomer as two integrase subunits rotate 180° relative to the other two subunits in a process known as subunit rotation. If the central dinucleotides of attP and attB sites are the same, the integrases ligate the newly aligned DNA strands together, thus completing the process. If the central dinucleotides are mismatched, they do not undergo ligation, and the integrases continue to rotate until the original attP and attB sites are restored (Bai et al., 2011; Rutherford et al., 2013; Smith, 2015).

Numerous studies have demonstrated the efficacy of large serine recombinases as genome engineering tools (Groth et al., 2004; Keravala et al., 2006; Low et al., 2022; Olivares et al., 2001; Russel et al., 2006; Thomason et al., 2001; Yamaguchi et al., 2011; Xu et al., 2008). They have been widely employed for targeted gene insertions in a variety of organisms, including bacteria, yeast, plants, and mammalian cells. These enzymes possess the remarkable capability to integrate large genetic payloads, ranging from several kilobases to several megabases, with high efficiency and accuracy.

However, a significant hurdle lies in the fact that integrases typically rely on predetermined target sites, which are not naturally present in the human genome or other genomes of interest. This limitation restricts their application for site-specific integration in complex biological systems.

In light of this challenge, researchers have resorted to a strategy that involves integrating an integrase wild-type target site into the genome and subsequently utilizing it as a landing platform for further genomic manipulations (Anzalone et al. 2022; Yarnall et al. 2023). This approach allows the subsequent integration of desired genetic payloads with increased specificity at the artificially created target sites. However, this method relies on complex machinery with multiple enzymes, which makes the whole construct difficult to introduce into cells with current delivery methods. Moreover, all different enzymes involved can contribute to additional off-target events.

Alternative approaches involve searching available metagenome datasets for enzymes with the required target site recognition (Durrant et al., 2023), or finding more flexible enzymes that can act on pseudo-sites. Pseudo-sites are target sites in the human genome that share some similarity to the natural target site of the enzyme and can thus be used for integration (Thyagarajan et al., 2001). However, search for integrases in the metagenome databases also requires detailed characterization of the new enzymes for each target site, which can be very resource- and time-consuming, provided that a suitable integrase is found at all. At the same time, repurposing the enzymes to recognize additional pseudo-sites leads to an increased risk for high off-target activity, limiting possible therapeutic applications.

To enable the direct and site-specific integration at user-defined target sequences, it is necessary to modify the large serine recombinases and change their target site specificity. However, little is known about the 3D structure and domains involved in DNA recognition, which poses a challenge for rational design. Modification of LSRs is further hampered by the complexity of their natural target sites, which are usually non-symmetrical and non-repetitive. Because the specificity defining rules are so obscure, there is a need in the art to develop a new unbiased and open method to enable the change of target site preferences of LSRs or to further improve activity and/or efficiency of LSRs. There is further a need for novel variants of large serine recombinases allowing the introduction of desired DNA sequences at specific sites into the genome of a subject.

### SUMMARY OF THE INVENTION

The objective underlying the present invention is solved by the provision of a method for generating a target-specific large serine recombinase (LSR), the method comprising the steps of:
a) generating a plurality of first variants of an LSR by introducing one or more mutations into a sequence encoding the LSR;
b) generating a library of expression vectors, wherein each expression vector comprises a first region comprising the sequence encoding one of the first variants of the LSR, and a second region comprising at least two first target sites of the LSR, wherein the two first target sites are different from each other;
c) introducing the library of expression vectors into host cells;
d) culturing the host cells and expressing the first variants of the LSR;
e) isolating plasmid DNA from the culture of host cells;
f) determining whether a portion of the second region on the expression vector between the two first target sites has been excised by the first variant LSR;
g) generating a plurality of second LSR variants by introducing one or more mutations into the sequence encoding those first LSR variants that excised the portion of the second region on the expression vector between the at least two first target sites;
h) repeating steps b) to f) optionally with at least one second target site, wherein the at least one second target site differs from the first target sites in at least one nucleotide.

According to one embodiment, the method further comprises the step of repeating steps g) and h), preferably wherein steps g) and h) are repeated at least twice to at least 30 times.

According to another embodiment, the LSR in step a) as basis for the plurality of first variant LSRs is a naturally occurring LSR.

According to one embodiment, the two target sites in the second region of the expression vector are spaced apart by at least 50 nucleotides.

According to one embodiment, the determining step f) comprises (i) sequencing of the first and the second region of the expression vector, or (ii) performing restriction digestion on the sequence of the expression vector comprising the two target sites followed by analysis of the digestion fragments.

According to a preferred embodiment, the method further comprises the step of removing inactive variants of the variant LSR from the library of expression vectors.

According to a preferred embodiment, the one or more mutations in the sequence encoding the LSR are introduced by random mutagenesis using error-prone PCR.

According to a further embodiment, the first region on the vector further comprises a unique molecular identifier.

According to one embodiment, the method further comprises one or more of the steps of:
(i) clustering the unique molecular identifiers,
(ii) generating and polishing consensus sequences,
(iii) determining the number of DNA modification events for each variant LSR enzyme;
(iv) determining an activity rate for each LSR variant; and/or
(v) selecting a target-specific LSR from the variant LSRs generated in step a) or steps g) to h) of claim 1 that excises the DNA sequence on the second region on the expression vector between two variant target sites of interest.

According to one preferred embodiment, the expression vector is a pEVO vector.

According to a further aspect, the present invention provides a variant large serine recombinase (LSR) obtainable or obtained by the method of the present invention, wherein the amino acid sequence of the variant LSR differs in at least one amino acid from the amino acid sequence of the LSR from which the variant is generated in step a), and from the amino acid sequence of any other naturally occurring large serine recombinase. According to a preferred embodiment, the variant comprises or consists of an amino acid sequence having at least 85% identity to one of SEQ ID NOs: 14, 17 to 35, 38 to 56, 60 to 8391, 8395, 8398 to 8403, 8406 to 8412, 8415 to 8422, 8425 to 8429 and, 8430 to 13115.

According to one embodiment, the variant LSR is a variant of an LSR selected from the group consisting of A118, TP901, φRV1 (also termed PhiRv1), Bxb1, φC31, R4, Wβ, Tnpx, Cp36, Dn29, Kp03, Nm60, Pa01, Si74.

According to a yet a further aspect, the present invention provides a nucleic acid or group of nucleic acids encoding a variant LSR according to the invention.

According to a yet a further aspect, the present invention provides an expression vector comprising a nucleic acid or group of nucleic acids of the present invention.

According to a yet a further aspect, the present invention provides a system for integrating a donor DNA into a target nucleic acid, the system comprising a polypeptide comprising a variant LSR according to the present invention or a nucleic acid encoding the same, and a donor nucleic acid to be inserted into the target nucleic acid.

According to a yet a further aspect, the present invention provides a pharmaceutical composition comprising the variant LSR of the invention, the nucleic acid or group of nucleic acids of the invention, or the expression vector of the invention, and optionally a pharmaceutically acceptable carrier.

According to a yet a further aspect, the present invention provides the use of the variant LSR of the invention, the nucleic acid or group of nucleic acids of the invention, the expression vector of the invention, the system of the invention, or of the pharmaceutical composition of the invention for integrating a nucleic acid sequence of interest into the genome of a subject or cell, wherein the cell preferably does not include cells of the human germ line.

According to a yet a further aspect, the present invention provides the variant LSR of the invention, the nucleic acid or group of nucleic acids of the invention, the expression vector of the invention, or the pharmaceutical composition of the invention for use in medicine.

According to a yet a further aspect, the present invention provides the variant LSR for use of the invention, the nucleic acid or group of nucleic acids for use of the invention, the expression vector for use of the invention, or the pharmaceutical composition for use of the invention, for use in the treatment of a genetic disease or disorder. The genetic disease or disorder is preferably a monogenetic disease or disorder. Further aspects and embodiments of the invention will become apparent from the appending claims and the following detailed description.

### DESCRIPTION OF THE DRAWINGS

The invention is further illustrated by the following figures and examples without being limited thereto.

FIG. 1 illustrates an integration reaction in nature. The bold circle represents the phage genome, with the phage attachment site attP. The line represents the bacterial genome, with the bacterial attachment site attB. In the presence of integrase, the integration reaction proceeds, resulting in the phage genome integrated into the bacterial chromosome, flanked by two hybrid att sites termed attL and attR.

FIG. 2A illustrates the domain organization of large serine recombinases.

FIG. 2B illustrates the structure of the LSR in the free unbound state.

FIG. 2C illustrates the mechanism of site-specific recombination.

FIGS. 3A-3C illustrate the evolution process of different integrases. FIG. 3A shows the process for TP901 integrase, FIG. 3B shows the process for PhiRv1 integrase, and FIG. 3C shows the process for A118 integrase.

FIGS. 4A-4B show the analysis of single clones from the evolved libraries of TP901 integrase.

FIG. 4A is the library evolved towards pRT798 target sites, and FIG. 4B is the library evolved towards target sites pRT799. Upper panel: agarose gel pictures showing the results of recombination test for single clones with 1 μg/ml arabinose induction. Pictograms at the right of the gel scheme indicate to which fragment each band corresponds to. Numbers at the bottom show recombination efficiency. Two triangles represent non-recombined fragments, and one triangle represents recombined fragments. M=molecular weight marker. Lower panel: sequence alignment of each clone with the wild-type enzyme (top line). Dot represents no change, * is a stop codon, # is an insertion. Positions of each mismatch are indicated in the bottom line.

FIGS. 5A-5B show the analysis of single clones from the evolved libraries of PhiRv1 integrase. FIG. 5A is the library evolved towards pRT824 target sites, and FIG. 5B is the library evolved towards target sites pRT825. Upper panel: agarose gel pictures showing the results of recombination test for single clones with 0, 1 and 10 μg/ml arabinose induction. Pictograms at the right of the gel scheme indicate to which fragment each band corresponds to. Two triangles represent non-recombined fragments, and one triangle represents recombined fragments. Lower panel: sequence alignment of each clone with the wild-type enzyme (top line). Dot represents no change, * is a stop codon. Positions of each mismatch are indicated in the bottom line.

FIGS. 6A-6C show the analysis of single clones from the evolved libraries of A118 integrase. FIG. 6A is the library evolved towards pRT783 target sites, FIG. 6B is the library evolved towards target sites pRT784, and FIG. 6C is the library evolved towards target sites pRT787. Upper panel: agarose gel pictures showing the results of recombination test for single clones with 0, 1 and 10 μg/ml arabinose induction. Pictograms at the right of the gel scheme indicate to which fragment each band corresponds to. Two triangles represent non-recombined fragments, and one triangle represents recombined fragments. Lower panel: sequence alignment of each clone with the wild-type enzyme (top line). Dot represents no change, * is a stop codon. Positions of each mismatch are indicated in the bottom line.

FIG. 7 shows the plasmid map of the pEVO4gg vector.

FIGS. 8A-8D illustrate the evolution process of Bxb1 integrase towards different target sites. FIG. 8A shows the process for the SH1 target site, FIG. 8B shows the process for the SH2 target site, FIG. 8C shows the process for the SH3 target site, and FIG. 8D shows the process for the SH4 target site.

FIGS. 9A-9B show the analysis of single clones from the libraries evolved towards SH2 target site. FIG. 9A: agarose gel pictures showing the results of recombination test for single clones with 0, 1 and 10 μg/ml arabinose induction. Numbers at the bottom show recombination efficiency. Pictograms at the right of the gel scheme indicate to which fragment each band corresponds to. Two triangles represent non-recombined fragments, and one triangle represents recombined fragments. M=molecular weight marker. FIG. 9B: sequence alignment of each clone with the wild-type enzyme (top line). Only positions with differences to the wild-type reference are shown. Dot represents no change, * is a stop codon. Positions of each mismatch are indicated in the bottom line.

FIGS. 10A-10B show the analysis of single clones from the libraries evolved towards the SH4 target site. FIG. 10A: agarose gel pictures showing the results of recombination test for single clones with 0, 1 and 10 μg/ml arabinose induction. Numbers at the bottom show recombination efficiency. Pictograms at the right of the gel scheme indicate to which fragment each band corresponds to. Two triangles represent non-recombined fragments, and one triangle represents recombined fragments. M=molecular weight marker. FIG. 10B: sequence alignment of each clone with the wild-type enzyme (top line). Only positions with differences to the wild-type reference are shown. Dot represents no change, * is a stop codon. Positions of each mismatch are indicated in the bottom line.

FIGS. 11A-11B show the analysis of single clones from the libraries evolved towards the SH3 target site. FIG. 11A: agarose gel pictures showing the results of recombination test for single clones with 0, 1 and 10 μg/ml arabinose induction. Numbers at the bottom show recombination efficiency. Pictograms at the right of the gel scheme indicate to which fragment each band corresponds to. Two triangles represent non-recombined fragments, and one triangle represents recombined fragments. M=molecular weight marker. FIG. 11B: sequence alignment of each clone with the wild-type enzyme (top line). Only positions with differences to the wild-type reference are shown. Dot represents no change, * is a stop codon. Positions of each mismatch are indicated in the bottom line.

FIGS. 12A-12B show the analysis of single clones from the libraries evolved towards the SH1 target site. FIG. 12A: agarose gel pictures showing the results of recombination test for single clones with 0, 1 and 10 μg/ml arabinose induction. Numbers at the bottom show recombination efficiency. Pictograms at the right of the gel scheme indicate to which fragment each band corresponds to. Two triangles represent non-recombined fragments, and one triangle represents recombined fragments. M=molecular weight marker. FIG. 12B: sequence alignments of each clone with the wild-type enzyme (top line). Only positions with differences to the wild-type reference are shown. Dot represents no change, * is a stop codon. Positions of each mismatch are indicated in the bottom line.

FIG. 13 sequence alignment of the clones from the Nanopore screen chosen for the evaluation in mammalian cell culture. The wild-type Bxb1 enzyme (top line) was used as reference. Only positions with differences to the wild-type reference are shown. Dot represents no change, * is a stop codon. Positions of each mismatch are indicated in the bottom line.

FIG. 14 shows the generalised plasmid map of the pCAGGS-attB-mCherry-attP-EGFP reporter vector.

FIG. 15 shows the generalised plasmid map of the EF1a-tagBFP-P2A-Int expression vector with integrase.

FIG. 16 shows a scheme of the plasmid-based assay used to quantify recombination efficiency of evolved integrase variants in mammalian cells. pCAGGS-attB-mCherry-attP-EGFP reporter plasmid encodes a red fluorescent protein mCherry flanked by tested target sites, followed by terminator sequences to stop transcription, and the EGFP gene, which is not expressed from the original plasmid. Upon recombination of the target sites, the mCherry cassette with the terminator sequence is excised, which leads to expression of a downstream EGFP gene.

FIG. 17 shows recombination efficiencies of single clones measured by using a reporter assay. The figure shows quantification of recombination efficiencies 48 h after transfection of the cells with indicated recombinases, analysed by flow cytometry (n=2, biological replicates are shown as dots). Error bars represent standard deviation of the mean (SD).

FIG. 18 shows recombination efficiencies of single clones nominated from Nanopore screens and measured with a reporter assay. Top panel shows recombination on SH4 target sites, lower panel shows recombination on SH3 target sites. N=2, biological replicates are shown as dots, error bars represent standard deviation of the mean (SD).

FIG. 19 shows gel analysis of PCR product evidencing the successful integration of a donor nucleic acid sequence into the SH2 locus using an evolved LSR.

### LIST OF SEQUENCES

The sequences referred to herein are disclosed in detail in the accompanying sequence listing. Exemplary and at the same time preferred sequences of the present invention are also listed in Tables 2 to 5 in the following detailed part of the description.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and it is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. Any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The “percentage of sequence identity” is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” is used herein in the context of two or more nucleic acids or polypeptide sequences, to refer to two or more sequences or subsequences that are the same, i.e. that comprise the same sequence of nucleotides or amino acids. Sequences are “identical” to each other if they have a specified percentage of nucleotides or amino acid residues that are the same. According to the present invention, at least 60% identical includes at least at least 61%, at least at least 62%, at least at least 63%, at least at least 64%, at least at least 65%, at least at least 66%, at least at least 67%, at least at least 68%, at least at least 69%, at least 70%, at least 71%, at least 72%, at least 73%, at least 74%, at least 75%, at least 76%, at least 77%, at least 78%, at least 79%, at least 80, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.2%, at least 99.5%, or at least 99.7% identity over the specified sequence, when compared and aligned for maximum correspondence over a comparison window, or designated region as measured using one of the following sequence comparison algorithms or by manual alignment and visual inspection. These definitions also refer to the complement of a test sequence. Accordingly, the term “at least XY % sequence identity” is used throughout the specification with regard to polypeptide and polynucleotide sequence comparisons. This expression preferably refers to a sequence identity of at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.2%, at least 99.5%, or at least 99.7% to the respective reference polypeptide or to the respective reference polynucleotide.

In the context of the present invention, a protein comprising an amino acid sequence having at least 80% identity to a given SEQ ID NO preferably means that said protein has an amino acid sequence having at least 85%, at least 90%, at least 92%, at least 94%, at least 96%, at least 98%, at least 99%, at least 99.2%, at least 99.5%, or at least 99.7% sequence identity to the given SEQ ID NO.

Likewise, in the context of the present invention, a nucleic acid sequence having at least 60% sequence identity to a given SEQ ID NO or a nucleic acid sequence reverse complementary thereto preferably means that said nucleic acid has a sequence having at least 65%, at least 70%, at least 75%, at least 80%, at least 85%, at least 90%, at least 92%, at least 94%, at least 96%, at least 98%, at least 99%, at least 99.2%, at least 99.5%, or at least 99.7% sequence identity to the given SEQ ID NO or a nucleic acid sequence reverse complementary to said SEQ ID NO.

The term “sequence comparison” is used herein to refer to the process wherein one sequence acts as a reference sequence, to which test sequences are compared. When using a sequence comparison algorithm, test and reference sequences are entered into a computer, if necessary, subsequence coordinates are designated, and sequence algorithm program parameters are designated. Default program parameters are commonly used, or alternative parameters can be designated. The sequence comparison algorithm then calculates the percent sequence identities for the test sequences relative to the reference sequence, based on the program parameters. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by one of the SEQ ID NOs of the present invention, if not specifically indicated otherwise.

Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch 1970, by the search for similarity method of Pearson and Lipman 1988, by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, 1989) alignments (B) of 50, expectation (E) of 10, M=5, N=−4, and a comparison of both strands. The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

The term “nucleic acid” and “nucleic acid molecule” are used synonymously herein and are understood as well-accepted in the art, i.e. as single or double-stranded oligo- or polymers of deoxyribonucleotide or ribonucleotide bases or both. The term “nucleic acids” as used herein includes not only deoxyribonucleic acids (DNA) and ribonucleic acids (RNA), but also all other linear polymers in which the bases adenine (A), cytosine (C), guanine (G) and thymine (T) or uracil (U) are arranged in a corresponding sequence (nucleic acid sequence). The invention also comprises the corresponding RNA sequences (in which thymine is replaced by uracil), complementary sequences and sequences with modified nucleic acid backbone or 3′ or 5′-terminus. Nucleic acids in the form of DNA are however preferred.

The term “large serine recombinase”, abbreviated as LSR, and the term “integrase” (including the term “integrase protein”) are used herein interchangeably and refer to an enzyme that is capable of manipulating the structure of a genome by integrating a nucleic acid into the genome of a subject or a cell. More specifically, the term refers to respective enzymes capable of catalyzing an integration reaction. LSRs are known in the art and described e.g. in Van Duyne et al., 2013, incorporated herein by reference. An integrase is preferably present in form of a monomer, a dimer or a tetramer, in particular in form of a dimer. Two integrase dimers usually work together to catalyze an integrase reaction. Dimers and tetramers can comprise two or four identical integrase protein monomers (homodimer or homotetramer), respectively, or alternatively two or more different monomers (heterodimer or heterotetramer).

The term “engineered DNA recombining enzyme” as used herein refers to any naturally occurring DNA recombining enzyme, preferably to an integrase protein, that has been further modified, e.g. evolved as described herein, in particular to modify its target-site specificity and/or its activity and/or efficiency.

The term “target site” (sometimes also referred to as “attachment site” or “att”) as used herein refers to a specific nucleotide sequence which an LSR recognizes, and at which DNA breakage and strand exchange occur. Naming of the sites is historically linked to their location: the attP site is found in the phage genome, and the attB site is present in the bacterial genome (Smith, 2015). These sequences typically range between 30 and 200 base pairs in length with the crossover site mostly centrally located and flanked by imperfect inverted repeats.

The term “target sequence” or “target nucleic acid” as used herein refers to a nucleic acid sequence comprising one or more target sites, such as the attB site, and on which an LSR dimer is formed for catalyzing an excision or integration reaction. For an integration reaction to occur, a donor sequence is integrated into the target sequence.

The term “donor sequence” or “donor nucleic acid” as used herein refers to a nucleic acid sequence to be integrated into the genome of a cell or a subject, and in particular into a target sequence thereof. The donor sequence comprises one or more target sites, such as the attP site, on which an LSR dimer is formed.

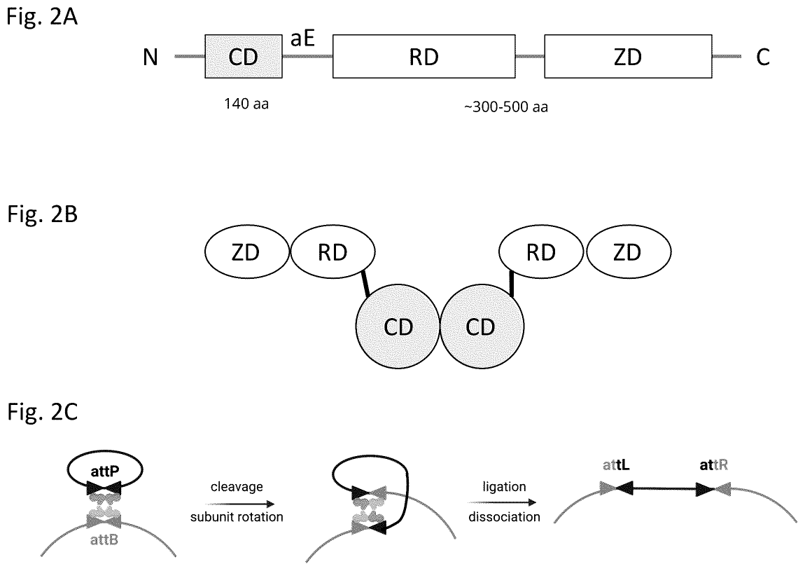

For a recombination event to occur, integrases bind to their target (attachment) sites on the target nucleic acid and on the donor nucleic acid as dimers, and then together form a tetramer, bringing the DNA sites together in a synaptic complex, which is illustrated in FIG. 2C. Sites forming after recombination are called attL (positioned left or upstream from the integration) and attR (positioned right or downstream from the integration), and contain half sites from attP and attB.

The term “therapeutically effective amount” as used herein, means that amount of active compound or pharmaceutical agent that elicits the biological or medicinal response in a tissue system, animal or human being sought by a researcher, veterinarian, medical doctor or other clinician, which includes alleviation of the symptoms of the disease or disorder being treated.

The term “pharmaceutical composition” as used herein refers to a substance and/or a combination of substances being used for the identification, prevention or treatment of a disease or tissue status. The pharmaceutical composition is formulated to be suitable for administration to a patient in order to prevent and/or treat a disease. Further, a pharmaceutical composition refers to the combination of an active agent with a carrier, inert or active, making the composition suitable for therapeutic use. Such a carrier is also referred to as being pharmaceutically acceptable. Pharmaceutical compositions can be formulated for oral, parenteral, topical, inhalative, rectal, sublingual, transdermal, subcutaneous or vaginal application routes according to their chemical and physical properties. Pharmaceutical compositions comprise solid, semisolid, liquid, transdermal therapeutic systems (TTS). Solid compositions are selected from the group consisting of tablets, coated tablets, powder, granulate, pellets, capsules, effervescent tablets or transdermal therapeutic systems. Also comprised are liquid compositions, selected from the group consisting of solutions, syrups, infusions, extracts, solutions for intravenous application, solutions for infusion or solutions of the carrier systems of the present invention. Semisolid compositions that can be used in the context of the invention comprise emulsion, suspension, creams, lotions, gels, globules, buccal tablets and suppositories.

As used herein, the term “pharmaceutically acceptable” embraces both human and veterinary use: For example, the term “pharmaceutically acceptable” embraces a veterinarily acceptable compound or a compound acceptable in human medicine and health care.

The term “subject” as used herein, refers to an animal, preferably a mammal, most preferably a human.

The term “plurality” as used herein includes any number of events described by an integer above one. The term “more than one” can be substituted for the term “plurality”.

The terms “LSR variant(s)” and “variant(s) of an/the LSR” are used interchangeably herein and denote an LSR differing from an LSR non-variant in at least one amino acid, for example an amino acid substitution, deletion or addition.

### Description of Embodiments

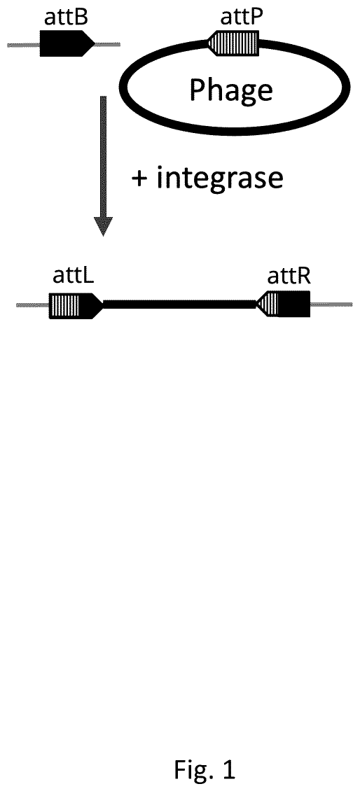

The present invention is in the field of large serine recombinases (LSRs). LSRs, also referred to as integrases, are known in the art and are described e.g. in Van Duyne et al., 2013, incorporated herein by reference. In contrast to tyrosine recombinases, large serine recombinases catalyze a unidirectional integration reaction. FIG. 1 schematically illustrates such an integration reaction. The bold circle represents the phage genome, with the phage attachment site attP. The line represents the bacterial genome, with the bacterial attachment site attB. In the presence of the integrase enzyme, the integration reaction proceeds, resulting in the phage genome becoming integrated into the bacterial chromosome, flanked by two hybrid att sites termed attL and attR.

The typical domain organization of large serine recombinases is illustrated in FIG. 2A. The catalytic domain (CD) is located at the N-terminal part of the protein, having a size of around 140 amino acids. In FIG. 2A, aE denotes the long alpha helix that connects the CD to the recombinase domain (RD) and the zinc ribbon domain (ZD). RD and ZD combined have a size of between approximately 300 and 500 amino acids. The structure of the LSR in its free unbound state is presented in FIG. 2B, showing the LSR as a compact and globular dimer, held together by interactions between catalytic domains. The typical mechanism of the integration reaction for LSRs is illustrated in FIG. 2C. The reaction starts when attachment site attP on the target sequence and attachment site attB on the donor sequence are each bound by an integrase dimer, followed by synapsis through intermolecular binding between both integrase dimers. DNA sequences are cleaved in the middle by the recombinase domain and rotated by 180°. The resulting sticky ends, which are complementary, are ligated. After rotation, the LSR dimers dissociate from the two newly generated attachment sites, attL and attR.

The present invention provides the first reliable and targeted method for either improving the activity and/or efficiency of large serine recombinases on their target sequences, or for changing the specificity of a large serine recombinase to essentially any desired target and donor sequences.

The present invention provides a method for generating a target-specific large serine recombinase (LSR), the method comprising the steps of:
a) generating a plurality of first variants of an LSR by introducing one or more mutations into a sequence encoding the LSR;
b) generating a library of expression vectors, wherein each expression vector comprises a first region comprising the sequence encoding one of the first variants of the LSR, and a second region comprising at least two first target sites of the LSR, wherein the two first target sites are different from each other;
c) introducing the library of expression vectors into host cells;
d) culturing the host cells and expressing the first variant LSR;
e) isolating plasmid DNA from the culture of host cells;
f) determining whether a portion of the second region on the expression vector between the two first target sites has been excised by the first variant LSR;
g) generating a plurality of second LSR variants by introducing one or more mutations into the sequence encoding those first LSR variants that excised the portion of the second region on the expression vector between the at least two first target sites;
h) repeating steps b) to f) optionally with at least one second target site, wherein the at least one second target site differs from the first target sites in at least one nucleotide.

According to one embodiment, the method further comprises the step of repeating steps g) and h), preferably wherein steps g) and h) are repeated at least twice to at least 30 times. That is, steps g) and h) are preferably repeated twice, three times, four times, five times, six times, seven times, eight times, nine times, ten times, eleven times, twelve times, 13×, 14×, 15×, 16×, 17×, 18×, 19×, 20×, 21×, 22×, 23×, 24×, 25×, 26×, 27×, 28×, 29×, 30× or more, wherein × denotes times.

In accordance with the present invention, a variant—be it the first, second, third or any subsequent variant—of an LSR differs from said known, initial or starting LSR in at least one amino acid. That is, the one or more mutations introduced into the sequence encoding the LSR leads to at least one or more amino acid substitution, deletion or addition, respectively, preferably to at least one or more amino acid substitution. Thus, according to a particularly preferred embodiment, the amino acid mutation is an amino acid substitution. Preferably, the variant differs from the known, initial or starting LSR in at least two, three, four, five, six, seven, eight, nine, ten, or more amino acids, preferably in at least 15, 20, 25, 30, 35, 40, 45, 50, 55, 60, 65, 70, 75, 80, 85, 90, 95, 100, 150, 200, 250, 300, 350, or 400 amino acids. For evolving LSRs, the first variant to be generated in accordance with a preferred embodiment of the present invention differs from a known LSR in at least one, two, three, four, five, six, seven, eight, nine, or ten or more amino acids, preferably in at least 15, 20, 25, 30, 35, 40, 45, 50, 55, 60, 65, 70, 75, 80, 85, 90, 95, or 100 amino acids. When repeating the step of generating LSR variants in step g) of the method, the new variant (also termed second variant) preferably differs from the initially generated or first LSR variant in at least one, two, three, four, five, six, seven, eight, nine, ten, or more amino acids, preferably in at least 15, 20, 25, 30, 35, 40, 45, 50, 55, 60, 65, 70, 75, 80, 85, 90, 95, or 100 amino acids. The same applies to each following LSR variant, which preferably differs from the previous LSR variant in at least one, two, three, four, five, six, seven, eight, nine, ten, or more amino acids, preferably in at least 15, 20, 25, 30, 35, 40, 45, 50, 55, 60, 65, 70, 75, 80, 85, 90, 95, or 100 amino acids. It is to be understood that the term “first LSR variant” denotes the starting LSR variant, and the term “second LSR variant” denotes the next and any further LSR variant generated. The term “second” is therefore not limited to only one subsequent LSR variant, but includes any following variant generated in accordance with each repeating of the method steps (step g)) of the present method, such as a third, fourth, fifth etc. variant. The method is thus not limited to just one cycle or repetition, but may include any number of cycles or repetitions necessary for generating the desired LSR variant. This may include at least two, three, four, five, six, seven, eight, nine, ten, eleven, twelve, 13, 14, 15, 16, 17, 18, 19, 20, 21, 22, 23, 24, 25, 26, 27, 28, 29, or at least 30 cycles or repetitions of the present method, preferably in accordance with step g) and optionally step h).

The difference in the amino acids may be a substitution, deletion or addition. A particularly preferred difference in amino acids in accordance with the variant LSRs described herein is an amino acid substitution. Hence, a variant LSR preferably differs from a known LSR or a previous variant LSR in that it contains at least one, two, three, four, five, six, seven, eight, nine, ten, or more amino acids, preferably in at least 15, 20, 25, 30, 35, 40, 45, 50, 55, 60, 65, 70, 75, 80, 85, 90, 95, or 100 amino acid modifications, preferably substitutions.

Variants of an LSR can be generated using any conventional method known in the art. According to a preferred embodiment, the variants are generated by amplifying the respective LSR gene with error-prone PCR to create a library of variants. According to a particularly preferred embodiment of the present invention, a low-fidelity DNA polymerase is used for error-prone PCR.

According to one particularly preferred embodiment, the LSR variants are based on a naturally occurring LSR. According to one such embodiment, the naturally occurring LSR is selected from the group consisting of but not limited to A118, TP901, φRV1, Bxb1, φC31, R4, Wβ, Tnpx, Cp36, Dn29, Kp03, Nm60, Pa01, and Si74. Thus, and in accordance with the present invention, for generating LSR variants based on a naturally occurring LSR, the nucleic acid sequence encoding said naturally occurring LSR, for example the coding sequence of A118, is subject to the method of the invention by introducing one or more mutations into the coding sequence thereof, e.g. by error-prone PCR as described herein. For generating LSR variants based on a non-naturally occurring LSR, the nucleic acid sequence encoding said non-naturally occurring LSR is subject to the method of the invention by introducing one or more mutations into the coding sequence thereof, e.g. by error-prone PCR as described herein. Thus, according to an alternative particularly preferred embodiment, the LSR variants are based on a non-naturally occurring LSR, such as a naturally-occurring LSR that has already been modified, or such as an artificially created LSR.

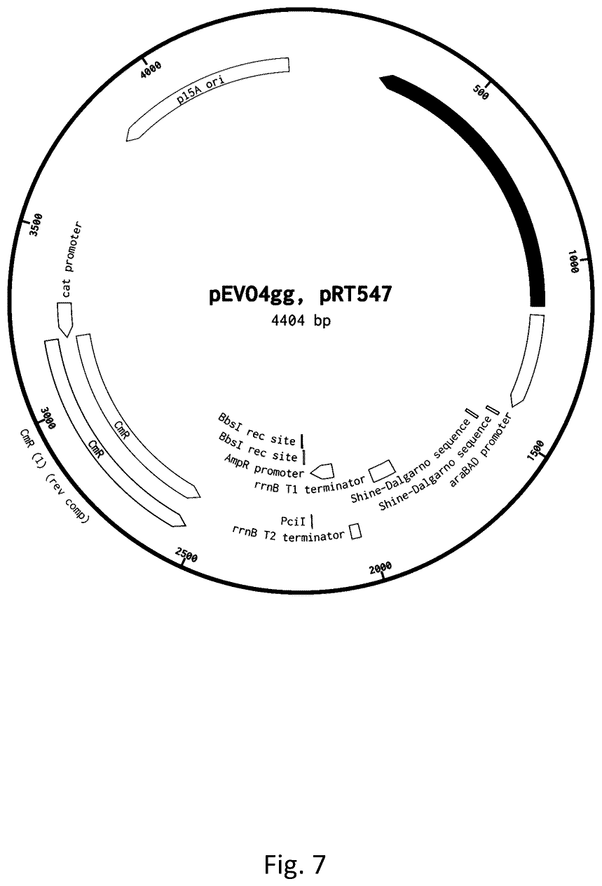

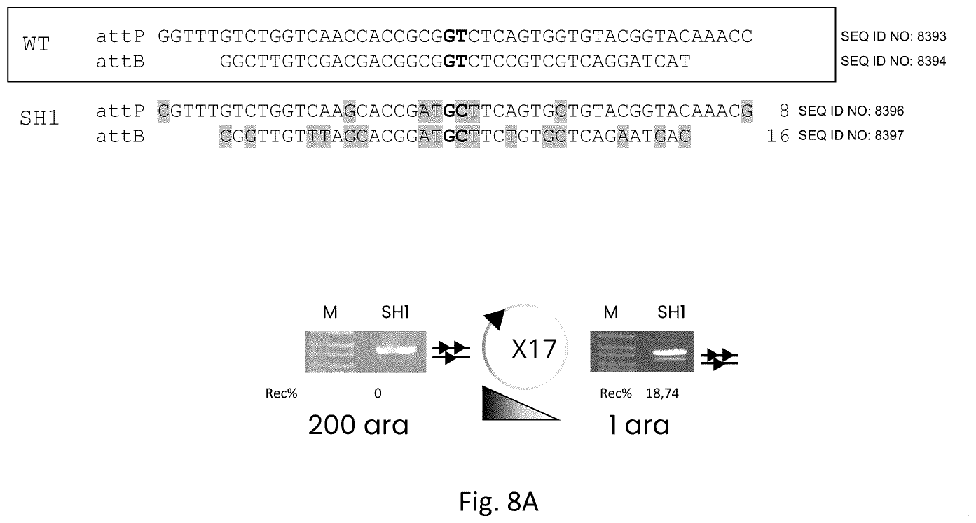

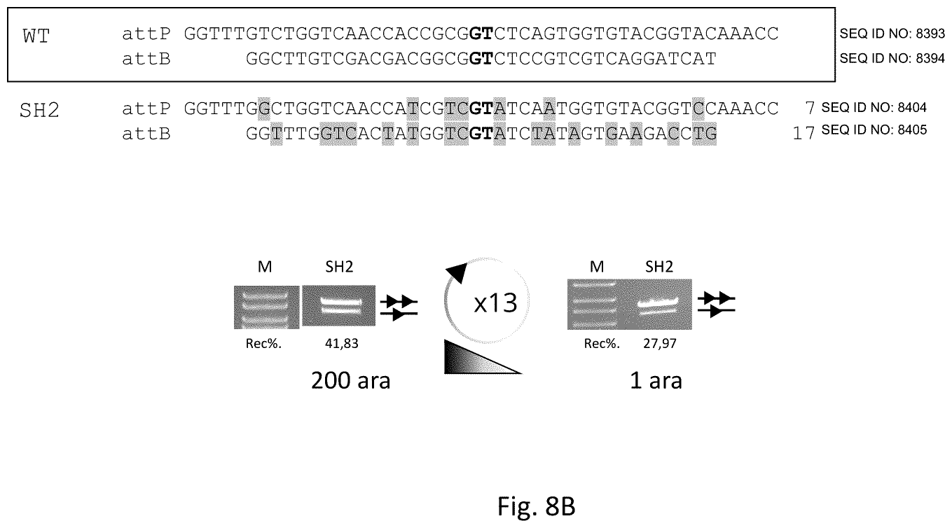

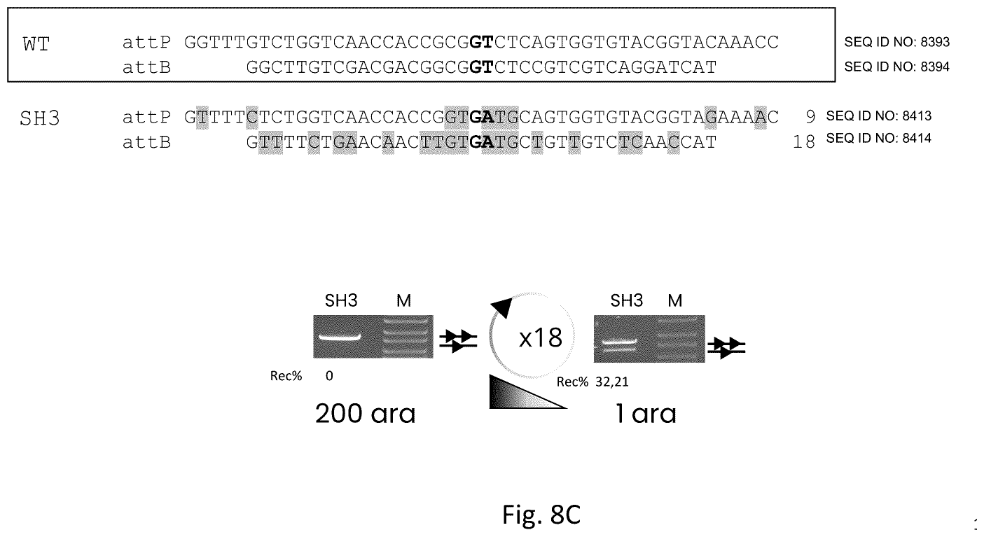

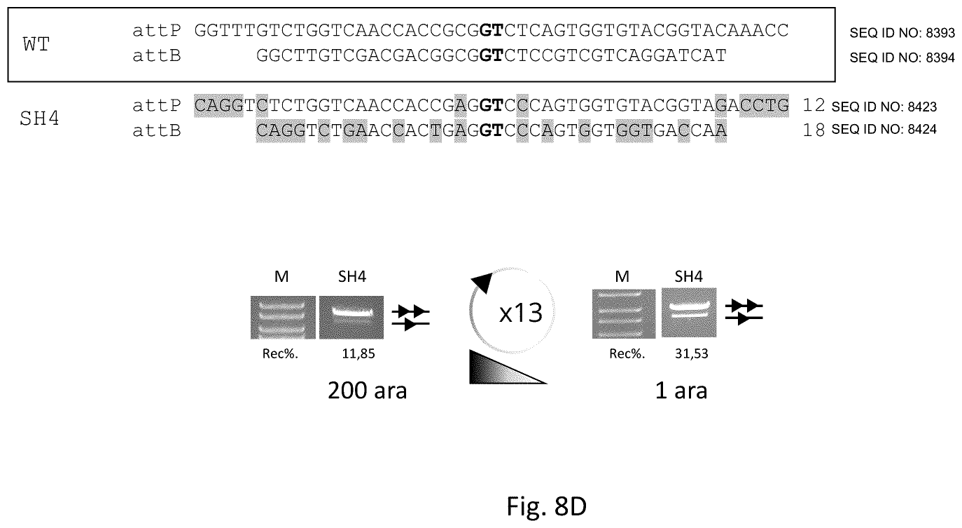

The mutated sequences encoding the variant LSRs, such as the PCR products of the error-prone PCR reaction, are introduced (e.g. cloned) into a suitable expression vector. This can be done by conventional methods such as digesting the coding nucleic acid using suitable restriction enzymes and ligating the coding nucleic acid into the expression vector. The expression vector to be used for the library of expression vectors can be any expression vector considered useful by the person of ordinary skill in the art. A preferred expression vector to be used in the context of the present invention is the pEVO expression vector described in Buchholz and Stewart 2001 or a modified version thereof described herein as pEVO4gg, shown in FIG. 7.

The expression vector comprises at least two regions. In the first region, the expression vector comprises the nucleic acid sequence encoding the LSR variant. According to a preferred embodiment, the first region further comprises a unique molecular identifier (UMI) for associating an identification means to each variant LSR. The term “unique molecular identifier” or “UMI” as used herein denotes a type of molecular barcoding. Molecular barcodes are short sequences to uniquely tag a molecule in a sample library. UMIs are known to the skilled person and are described in detail in Zurek et al., 2020, or Karst et al., 2021, both of which are herein incorporated by reference in their entirety. According to a preferred embodiment of the present invention, a UMI is an oligonucleotide comprising at least 5, at least 6, at least 7, at least 8, at least 9, at least 10, at least 20, at least 30, at least 40, at least 50, at least 60, at least 70, at least 80, at least 90 or at least 100 random nucleotides. According to a particularly preferred embodiment, a UMI is an oligonucleotide comprising at least 50 random nucleotides. A UMI may preferably form part of a UMI-tag, which may further comprise one or more sequences down-stream and/or upstream of the random nucleotides of the UMI (e.g. flanking the random nucleotides), which sequences may serve as one or more primer binding sites. A UMI-tag may further comprise one or more and preferably at least two restriction sites preferably down-stream and/or upstream of the random nucleotides of the UMI. According to a preferred embodiment, a UMI-tag comprises a first primer binding site, a first restriction site, the random nucleotides of the UMI, a second restriction site, and a second primer binding site.

In addition to the sequence encoding the variant LSR in the first region of the expression vector, the expression vector further comprises a second region comprising at least two first target sites. The second region on the expression vector is separated from the first region in that both regions do not overlap.

Naturally occurring LSR target sites differ from each other. Thus, according to one embodiment of the present invention, the target sites differ from each other, preferably in at least one, two, three, four, five, six, seven, eight, nine, ten, eleven, twelve, 13, 14, 15, 16, 17, 18, 19, 20, 21, 22, 23, 24, 25, 26, 27, 28, 29 or at least 30 nucleotides. Such a difference may also include the length of the target sites, if both are compared to each other. Depending on the intended direction of evolution of the LSRs, each of the second target sites may differ from the respective first target site in at least one, two, three, four, five, six, seven, eight, nine, ten, eleven, twelve, 13, 14, 15, 16, 17, 18, 19, 20, 21, 22, 23, 24, 25, 26, 27, 28, 29 or at least 30 nucleotides. According to a further embodiment, only one of the second target sites differs from the respective first target site in at least one, two, three, four, five, six, seven, eight, nine, ten, eleven, twelve, 13, 14, 15, 16, 17, 18, 19, 20, 21, 22, 23, 24, 25, 26, 27, 28, 29 or at least 30 nucleotides, and the other one of the second target sites does not differ from the respective first target site.

The first target sites are preferably the target sites of the respective LSR to be mutated in step a) of the method of the present invention. For example, in cases of a naturally occurring LSR to be the starting point of the method of the present invention and thus the template for the mutations in step a), the at least two target sites can be those of the naturally occurring LSR. In such a case, the variant LSR is evolved to have an increased specificity to the first target sites and/or increased activity (also referred to as increased efficiency) on the first target sites compared to the respective naturally occurring LSR. Such an increase in specificity and/or activity is preferably at least 10%, 20%, 30%, 40%, 50%, 60%, 70%, 80%, 90%, 100%, 150%, 200%, 250%, or at least 300% compared to the starting/initial LSR. The same applies to the generation of subsequent variant LSRs, which are based on a previous variant LSR.

A table showing naturally occurring LSRs (also referred to as wildtype LSRs) used for the generation of LSR variants, and their respective natural target sites is presented below in Table 1.

TABLE 1
Wildtype LSRs and their target sites.

| | Amino acid | First target | Second target |
|---|---|---|---|
| LSR | sequence | site (P) | site (B) |
| TP901-1 | SEQ ID NO: 5 | SEQ ID NO: 6 | SEQ ID NO: 7 |
| PhiRv1 | SEQ ID NO: 8 | SEQ ID NO: 9 | SEQ ID NO: 10 |
| A118 | SEQ ID NO: 11 | SEQ ID NO: 12 | SEQ ID NO: 13 |
| Bxb1 | SEQ ID | SEQ ID | SEQ ID |
| | NO: 8392 | NO: 8393 | NO: 8394 |

According to one preferred embodiment of the present invention, for generating LSRs that are active on a desired target site (also termed a target site of interest), one or both of the at least two first target sites are variants of the respective target sites of the LSR, from which the variants are generated in step a) of the method of the present invention. According to one embodiment, one or both of the at least two first target sites differ from the respective target sites of the LSR in at least one, two, three, four, five, six, seven, eight, nine, ten, eleven, twelve, 13, 14, 15, 16, 17, 18, 19, 20, 21, 22, 23, 24, 25, 26, 27, 28, 29 or at least 30 nucleotides. Alternatively, any nucleic acid sequence of interest could be used as target site in the method of the invention. The present invention thus allows generating LSRs which are active on desired target sites, preferably on target sites present in the genome of an organism such as a mammal and more preferably of a human. Particularly preferred target sites are those in the genome of a subject, preferably a human, which are located in safe harbor sites (SHS). SHS and methods for their identification are known in the respective field and are described e.g. in Aznauryan et al., 2022. For applications that do not require precise targeting of an existing gene or locus (e.g. to introduce or modify an endogenous gene, allele, or regulatory element), a common strategy is to target transgene integration to one of a small number of chromosomal SHS for expression, presumably without disrupting the expression of adjacent or more distant genes. Known SHS include but are not limited to the AAVS1 site on chromosome 19q and those described in Pellenz et al., 2019, incorporated herein by reference. Other preferred target sites are selected such that an endogenous gene, allele, or regulatory element can be modified by inserting a respective donor nucleic acid.

According to a preferred embodiment of the present invention, the at least two target sites in the second region on the expression vector are spaced apart from each other by at least about 50, 55, 60, 65, 70, 75, 80, 85, 90, 95, 100, 110, 120, 130, 140, 150, 160, 170, 180, 190, or at least about 200 nucleotides. According to a particularly preferred embodiment, the at least two target sites in the second region on the expression vector are spaced apart from each other by at least about 50 nucleotides, more preferably by at least about 100 nucleotides.

The library of expression vectors containing the nucleotide sequences encoding the variant LSRs and the target sites are subsequently introduced into suitable (host) cells. The method according to the invention can be performed using eukaryotic and prokaryotic (host) cells. Preferred prokaryotic cells are bacterial cells. Particularly preferred prokaryotic cells are cells of Escherichia coli. Preferred eukaryotic cells are yeast cells (preferably Saccharomyces cerevisiae), insect cells, non-insect invertebrate cells, amphibian cells, or mammalian cells (preferably somatic or pluripotent stem cells, including embryonic stem cells and other pluripotent stem cells, like induced pluripotent stem cells, and other native cells or established cell lines, including NIH3T3, CHO, HeLa, HEK293, hiPS). According to a particularly preferred embodiment, the (host) cells are XL-1 Blue E. coli cells, and the ligated plasmids are introduced via electroporation of the cells. The skilled person is well aware about alternative suitable methods for introducing a ligated plasmid into a host cell for subsequent expression of the encoded protein. According to one embodiment, the cell is not a human germ cell.

The host cells carrying the library of expression vectors are cultured to allow the expression of the encoded LSR variants. The culturing conditions are not particularly limited and will be selected by the skilled person based on the host cells used. For example, in case of using XL-1 Blue E. coli cells, it is preferred to culture the transformed bacteria in LB medium at 37° C. Conditions for introducing expression of the encoded LSR variants also depend on the host cells and plasmid vectors used. In the case of using pEVO expression vectors and XL-1 Blue E. coli cells, expression can be induced by adding e.g. arabinose to the culture medium.

Although LSRs are known to integrate a donor DNA into a genome, the expressed LSR variants—if active on the respective target sites—actively excise the portion (i.e. the nucleotide sequence) between the at least two target sites on the expression vector without integrating any DNA.

After cultivation and induction of expression, plasmid DNA of these cultures is isolated using any suitable method known to the person or ordinary skill in the art. The isolated plasmid DNA can then be analysed for activity of the LSR variant on the target sites. For example, at least the respective portion of the plasmid DNA encoding the at least two target sites and any nucleotides in between the at least two target sites can be sequenced. Thus, according to one embodiment, the step of determining whether the portion of the second region of the expression vector between the at least two target sites has been excised by the variant LSR involves sequencing of at least the second region of the expression vector. The sequencing may also include the first region of the expression vector encoding the variant LSR. Alternatively, the plasmid DNA may be digested using one or more restriction enzymes. Thus, according to one embodiment, the step of determining whether the portion of the second region on the expression vector between the at least two target sites has been excised by the variant LSR involves performing restriction digestion on the sequence of the expression vector comprising the two target sites, followed by analysis of the digestion fragments. The restriction enzyme(s) is preferably selected so as to excise the portion of the plasmid encoding the variant LSR, leading to a larger fragment including the sequence in between the at least two target sites in case of no excision by the LSR (that is the LSR is inactive on the target sites), and to a smaller fragment in case of an excision reaction between the at least two target sites (that is the LSR is active on the target sites). When determining whether the variant LSR is active on the target sites, those variants that turned out not to be able to excise the portion between the at least two target sites will be removed from the library. Thus, according to one embodiment of the present invention, the method further comprises the step of removing inactive LSR variants from the library of expression vectors. In such cases, the removed variants will not be included in method steps g) and h). An exemplary method of removing inactive LSR variants is analogous to the restriction digestion method for determining whether the LSR variant is active on the respective target sites. The restriction enzyme(s) digest the plasmid between the target sites encoded in the second region of the vector. In the absence of an excision reaction, the restriction enzyme will cut the vector in between the two target sites and the vector will be linearized. If the LSR is active, the respective portion between the two target sites will be excised and no restriction digestion that could linearize the plasmid will occur. PCR primers are selected so as to allow amplification of the first region comprising the sequence encoding the variant LSR and the second region comprising the at least two first target sites, wherein the PCR primers point at each other. If an excision reaction took place, any restriction site is removed and the vector stays intact, preserving correct orientation the PCR primers and thus amplification of a product including the first and the second regions on the plasmid. This PCR product can then be used for the next evolution cycle in that it serves as basis for introducing further mutations e.g. by error-prone PCR as described for step a) to generate second LSR variants.

According to the present invention, step a) is repeated with those first active LSR variants by introducing one or more mutations into the sequence encoding these first LSR variants to generate second LSR variants. This step can be performed analogous to step a) with the variant LSRs of the first round as basis for the mutations instead of the naturally occurring LSRs.

Likewise, method steps b) to f) are repeated. For generating LSR variants with improved efficiency/activity on the target sites, the target sites may be used unchanged, i.e. with the same nucleotide sequences as in the previous round or cycle. If the LSR variants shall be evolved in the direction of variant target sites differing from the first target sites used, at least one target site used in step b) may optionally be modified. Such variant second target site(s) may differ from the previously used first target sites in at least one, two, three, four, five, six, seven, eight, nine, ten, eleven, twelve, 13, 14, 15, 16, 17, 18, 19, 20, 21, 22, 23, 24, 25, 26, 27, 28, 29 or at least 30 nucleotides.

The method steps disclosed herein may be repeated until a variant LSR is generated with an improved efficiency/activity on naturally occurring target sites or having activity on desired target sites (i.e. having a changed specificity). The method for determining the activity and or specificity of LSRs is not particularly limited. According to one particularly preferred embodiment, activity is determined by performing restriction digestion on the sequence of the expression vector comprising the two target sites, followed by analysis of the digestion fragments. A larger fragment is obtained in case of no excision reaction by the LSR, and a smaller fragment is obtained in case of an excision reaction by the LSR between the at least two target sites. Comparing the amount of the smaller fragment with the amount of the larger fragment allows the calculation of a percentage value, which reflects the activity of the LSR on the respective target sites. In more detail, the sequence encoding an LSR or LSR variant is preferably introduced into an expression vector comprising the respective target sites and/or variant target sites. The expression vectors are then introduced into suitable cells such as bacteria cells, which are cultured under conditions allowing the expression of the encoded protein (e.g. by adding e.g. arabinose to the culture medium). Plasmid DNA may then be isolated and digested using one or more restriction enzymes. The restriction enzyme(s) is preferably selected so as to excise the portion of the plasmid encoding the target sites and optionally the variant LSR, leading to a larger fragment including the sequence in between the at least two target sites in case of no excision reaction by the LSR, and to a smaller fragment in case of an excision between the at least two target sites. The size difference can be visualized for example in gel electrophoresis. The visualizations can then be analysed for the relative amount of large and small fragments, allowing the calculation of a percentage value for recombined and non-recombined (excised and non-excised) plasmids. Band intensity values can for example be divided by the combined values of the recombined and non-recombined bands to determine the fraction of recombined DNA, which can be converted to a percentage value by multiplying with 100.

An alternative method for determining whether or not an LSR is active on the target sites in the expression vector is by analysing the nucleic acid sequence of the vector in the isolated plasmids. This can be done by PCR amplification of the region comprising the target sites and optionally the region encoding the LSR variant, and/or by sequencing the region comprising the target sites and optionally the region encoding the LSR variant. In cases of an excision reaction by an active LSR, the sequence will be shorter, lacking the excised sequence between the two target sites.

According to a preferred embodiment, an active LSR exhibits at least about 10% activity, preferably at least about 15%, about 20%, about 25%, about 30%, about 35%, about 40%, about 45%, or at least about 50% activity on the respective target sites. According to a preferred embodiment, LSRs that show less than about 1%, less than about 2%, less than about 3%, less than about 4%, or less than about 5% activity, are considered as being inactive on the respective target sites.

Further methods for determining the activity of an enzyme are well known in the art. One such further and preferred method for determining activity of an LSR comprises digesting recombined and non-recombined plasmids from the culture of host cells with restriction enzymes that excise the LSR gene. Due to differences in plasmid sizes between the two versions, the resulting DNA fragments containing the plasmid backbone also show a difference in size. The size difference can be visualized for example in agarose gel electrophoresis. The visualizations can then be analysed for the relative amount of large and small fragments, allowing the calculation of a percentage value for recombined and non-recombined plasmids. Band intensity values can for example be divided by the combined values of the recombined and non-recombined bands to determine the fraction of recombined DNA, which can be converted to a percentage value by multiplying with 100.

According to a further embodiment, the method of the present invention further comprises the step of clustering the unique molecular identifiers, if any are present in the first region of the expression vector. According to a further embodiment, the method of the present invention further comprises the step of generating and polishing consensus sequences. Clustering and polishing of respective sequencing results are described in Zurek at el., 2020, and Karst et al., 2021, both of which are incorporated herein by reference in their entirety. For clustering, the sequence at least in the first region of the expression vector encoding the variant LSR and preferably the UMI are sequenced by methods known in the art. The collected UMI sequences are then clustered based on sequence identity, while maintaining their association to their read identification (read IDs), i.e. the UMI. This can be done by any conventional method known in the art, such as by using VSEARCH (Rognes et al., 2016). Based on these clusters and their associated read IDs, the sequence reads can then be separated into different files, where each file contains reads that corresponds to one UMI-cluster. From each of these clusters of separated reads, the part of the sequence that contains the gene for the LSR variant is aligned to a reference gene that contains a high similarity to the screened variants. Typically, such a gene is determined through sequencing of the screened LSR variant library or by selecting the LSR variant gene that was modified to produce the screened variant library. With the aid of this alignment, a software for consensus sequence generation and sequence polishing can be used to determine the most likely gene sequence of the LSR variant associated to this cluster based on all sequence reads. Polishing in this respect refers to additional rounds of processing of the reads, leading to improved sequence accuracy.

According to a further embodiment, the method of the present invention further comprises the step of determining the number of DNA modification events for or in each variant LSR. This step identifies which amino acids in the variant LSR have been modified compared to the naturally occurring starting LSR or to the previous LSR from which the variant LSR is derived.

According to a further embodiment, the method of the present invention further comprises the step of determining an activity rate for each LSR variant generated by the method of the present invention. In particular, the activity rate can be determined for those LSR variants that have been found to excise the DNA sequence on the second region on the expression vector between the at least two target sites of particular interest.

According to a further embodiment, the method of the present invention further comprises the step of selecting a target-specific LSR from the variant LSRs generated in step a) or steps g) to i) that excises the DNA sequence on the second region on the expression vector between two variant target sites of interest.

The present invention is also directed to variant LSRs (also termed LSR variants) obtained by the method of the present invention. These variant LSRs preferably differ in at least one amino acid, in at least two, three, four, five, six, seven, eight, nine, ten, or more amino acids, preferably in at least 15, 20, 25, 30, 35, 40, 45, 50, 55, 60, 65, 70, 75, 80, 85, 90, 95, 100, 150, 200, 250, 300, 350, or in at least 400 amino acids from any naturally occurring LSR, in particular from the LSRs A118, TP901, φRV1, φC31, R4, Wβ, Tnpx, Cp36, Dn29, Kp03, Nm60, Pa01, and Si74. The variant LSRs further preferably differ in at least one, in at least two, three, four, five, six, seven, eight, nine, ten, or more amino acids, preferably in at least 15, 20, 25, 30, 35, 40, 45, 50, 55, 60, 65, 70, 75, 80, 85, 90, 95, 100, 150, 200, 250, 300, 350, or in at least 400 amino acids from the LSR from which the first variant is generated in step a) of the method of the present invention. According to a particular preferred embodiment, the variant LSR differs from any LSR known to date (such as those described in Durrant et al., 2022) in at least one, in at least two, three, four, five, six, seven, eight, nine, ten, or more amino acids, preferably in at least 15, 20, 25, 30, 35, 40, 45, 50, 55, 60, 65, 70, 75, 80, 85, 90, 95, 100, 150, 200, 250, 300, 350, or in at least 400 amino acids. The difference between the LSR variant obtained by the method of the present invention and a naturally occurring LSR, in particular A118, TP901, φRV1, Bxb1, φC31, R4, Wβ, Tnpx, Cp36, Dn29, Kp03, Nm60, Pa01, and Si74, can also be expressed in terms of percentage identity. Accordingly, according to one embodiment, the LSR variant obtained by the method of the present invention has a sequence identity of not more than 80%, 81%, 82%, 83%, 84%, 85%, 86%, 87%, 88%, 89%, 90%, 91%, 92%, 93%, 94%, 95%, 96%, 97%, 98%, 99%, 99.2%, 99.5% or 99.7% to a naturally occurring LSR, in particular to A118, TP901, φRV1, Bxb1, φC31, R4, Wβ, Tnpx, Cp36, Dn29, Kp03, Nm60, Pa01, and Si74. According to a particular preferred embodiment, the LSR variant obtained by the method of the present invention has a sequence identity of between 80% and 90% to a naturally occurring LSR, in particular to A118, TP901, φRV1, φC31, R4, Wβ, Tnpx, Cp36, Dn29, Kp03, Nm60, Pa01, and Si74, more preferably of between 82% and 88%, even more preferably of between 84% and 86%, and most preferably of 85%. Such LSR variants are preferably active LSRs as defined herein.

According to a particularly preferred embodiment, the difference in the amino acid sequence is caused by one or more amino acid substitutions, i.e. the variant differs from the wildtype or the preceding variant in that it comprises a respective number of amino acid substitutions and thus the same overall number of amino acids as in the wildtype or the preceding variant.

Particularly preferred LSRs in accordance with the present invention comprise or consist of an amino acid sequence having at least 85% sequence identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115. More specifically preferred are LSRs that comprise or consist of an amino acid sequence having at least 85% sequence identity to any one of SEQ ID NOs: 14, 17 to 35, 38 to 56, 60 to 88, and 12437. According to a preferred embodiment, the LSRs in accordance with the present invention comprise or consist of an amino acid sequence having at least 86%, at least 87%, at least 88%, at least 89%, at least 90%, at least 91%, at least 92%, at least 93%, at least 94%, at least 95%, at least 96%, at least 97%, at least 98%, at least 99%, at least 99.2%, at least 99.5%, at least 99.7%, or 100% sequence identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115. According to a particularly preferred embodiment, the LSR consists of an amino acid sequence as set forth in one of SEQ ID NOs: 14, 17 to 35, 38 to 56, 60 to 8391, 8395, 8398 to 8403, 8406 to 8412, 8415 to 8422, 8425 to 8429, and 8430 to 13115, more preferably of an amino acid sequence as set forth in one of SEQ ID NOs: 14, 17 to 35, 38 to 56, 60 to 88, and 12437. According to one particular embodiment, the LSRs in accordance with the present invention comprise or consist of an amino acid sequence having at least 85% sequence identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115 are not identical to any large serine recombinase known in the art. All of these LSRs are preferably active LSRs as defined herein.

The present invention also provides fragments of the LSRs disclosed herein or obtained by the method of the present invention, in particular fragments of an LSR comprising or consisting of an amino acid sequence having at least 85% sequence identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115. In accordance with the present invention, a fragment LSRs may comprise an N-terminal and/or C-terminal deletion of one or more amino acids. According to a preferred embodiment, the deletion may comprise one, two, three, four, five, six, seven, eight, nine, ten, eleven, twelve, 13, 14, 15, 16, 17, 18, 19, 20, 21, 22, 23, 24, 25, 26, 27, 28, 29 or 30 amino acids compared to the non-fragment LSR. All of these LSRs are preferably active LSRs as defined herein.

The following tables list some generated variant LSRs and their respective target sequences. Thus, according to a particularly preferred embodiment of the present invention, an LSR or LSR variant according to the invention is selected from the LSR variants disclosed in Tables 2 to 5.

TABLE 2
TP901 LSR variants and their target sites.

| LSR | Amino acid | First target | Second target |
|---|---|---|---|
| variant | sequence | site (P) | site (B) |
| 798 cl2 | SEQ ID NO: 14 | SEQ ID NO: 6 | SEQ ID NO: 15 |
| 798 cl6 | SEQ ID NO: 17 | SEQ ID NO: 6 | SEQ ID NO: 15 |
| 798 cl10 | SEQ ID NO: 18 | SEQ ID NO: 6 | SEQ ID NO: 15 |
| 798 cl3 | SEQ ID NO: 19 | SEQ ID NO: 6 | SEQ ID NO: 15 |
| 798 cl4 | SEQ ID NO: 20 | SEQ ID NO: 6 | SEQ ID NO: 15 |
| 798 cl9 | SEQ ID NO: 21 | SEQ ID NO: 6 | SEQ ID NO: 15 |
| 798 cl7 | SEQ ID NO: 22 | SEQ ID NO: 6 | SEQ ID NO: 15 |
| 798 cl5 | SEQ ID NO: 23 | SEQ ID NO: 6 | SEQ ID NO: 15 |
| 798 cl8 | SEQ ID NO: 24 | SEQ ID NO: 6 | SEQ ID NO: 15 |
| 799 cl10 | SEQ ID NO: 25 | SEQ ID NO: 6 | SEQ ID NO: 16 |
| 799 cl5 | SEQ ID NO: 26 | SEQ ID NO: 6 | SEQ ID NO: 16 |
| 799 cl9 | SEQ ID NO: 27 | SEQ ID NO: 6 | SEQ ID NO: 16 |
| 799 cl3 | SEQ ID NO: 28 | SEQ ID NO: 6 | SEQ ID NO: 16 |
| 799 cl7 | SEQ ID NO: 29 | SEQ ID NO: 6 | SEQ ID NO: 16 |
| 799 cl1 | SEQ ID NO: 30 | SEQ ID NO: 6 | SEQ ID NO: 16 |
| 799 cl2 | SEQ ID NO: 31 | SEQ ID NO: 6 | SEQ ID NO: 16 |
| 799 cl8 | SEQ ID NO: 32 | SEQ ID NO: 6 | SEQ ID NO: 16 |
| 799 cl6 | SEQ ID NO: 33 | SEQ ID NO: 6 | SEQ ID NO: 16 |
| 799 cl4 | SEQ ID NO: 34 | SEQ ID NO: 6 | SEQ ID NO: 16 |

TABLE 3
PhiRv1 LSR variants and their target sites.

| LSR | Amino acid | First target | Second target |
|---|---|---|---|
| variant | sequence | site (P) | site (B) |
| 824 cl1 | SEQ ID NO: 35 | SEQ ID NO: 9 | SEQ ID NO: 36 |
| 824 cl8 | SEQ ID NO: 38 | SEQ ID NO: 9 | SEQ ID NO: 36 |
| 824 cl5 | SEQ ID NO: 39 | SEQ ID NO: 9 | SEQ ID NO: 36 |
| 824 cl10 | SEQ ID NO: 40 | SEQ ID NO: 9 | SEQ ID NO: 36 |
| 824 cl2 | SEQ ID NO: 41 | SEQ ID NO: 9 | SEQ ID NO: 36 |
| 824 cl3 | SEQ ID NO: 42 | SEQ ID NO: 9 | SEQ ID NO: 36 |
| 824 cl7 | SEQ ID NO: 43 | SEQ ID NO: 9 | SEQ ID NO: 36 |
| 824 cl4 | SEQ ID NO: 44 | SEQ ID NO: 9 | SEQ ID NO: 36 |
| 824 cl6 | SEQ ID NO: 45 | SEQ ID NO: 9 | SEQ ID NO: 36 |
| 824 cl9 | SEQ ID NO: 46 | SEQ ID NO: 9 | SEQ ID NO: 36 |
| 825 cl4 | SEQ ID NO: 47 | SEQ ID NO: 9 | SEQ ID NO: 37 |
| 825 cl1 | SEQ ID NO: 48 | SEQ ID NO: 9 | SEQ ID NO: 37 |
| 825 cl8 | SEQ ID NO: 49 | SEQ ID NO: 9 | SEQ ID NO: 37 |
| 825 cl9 | SEQ ID NO: 50 | SEQ ID NO: 9 | SEQ ID NO: 37 |
| 825 cl3 | SEQ ID NO: 51 | SEQ ID NO: 9 | SEQ ID NO: 37 |
| 825 cl10 | SEQ ID NO: 52 | SEQ ID NO: 9 | SEQ ID NO: 37 |
| 825 cl2 | SEQ ID NO: 53 | SEQ ID NO: 9 | SEQ ID NO: 37 |
| 825 cl7 | SEQ ID NO: 54 | SEQ ID NO: 9 | SEQ ID NO: 37 |
| 825 cl5 | SEQ ID NO: 55 | SEQ ID NO: 9 | SEQ ID NO: 37 |

TABLE 4
A118 LSR variants and their target sites.

| LSR | Amino acid | First target | Second target |
|---|---|---|---|
| variant | sequence | site (P) | site (B) |
| 783 cl7 | SEQ ID NO: 56 | SEQ ID NO: 12 | SEQ ID NO: 57 |
| 783 cl1 | SEQ ID NO: 60 | SEQ ID NO: 12 | SEQ ID NO: 57 |
| 783 cl2 | SEQ ID NO: 61 | SEQ ID NO: 12 | SEQ ID NO: 57 |
| 783 cl8 | SEQ ID NO: 62 | SEQ ID NO: 12 | SEQ ID NO: 57 |
| 783 cl10 | SEQ ID NO: 63 | SEQ ID NO: 12 | SEQ ID NO: 57 |
| 783 cl3 | SEQ ID NO: 64 | SEQ ID NO: 12 | SEQ ID NO: 57 |
| 783 cl6 | SEQ ID NO: 65 | SEQ ID NO: 12 | SEQ ID NO: 57 |
| 783 cl4 | SEQ ID NO: 66 | SEQ ID NO: 12 | SEQ ID NO: 57 |
| 783 cl9 | SEQ ID NO: 67 | SEQ ID NO: 12 | SEQ ID NO: 57 |
| 783 cl5 | SEQ ID NO: 68 | SEQ ID NO: 12 | SEQ ID NO: 57 |
| 784 cl9 | SEQ ID NO: 69 | SEQ ID NO: 12 | SEQ ID NO: 58 |
| 784 cl1 | SEQ ID NO: 70 | SEQ ID NO: 12 | SEQ ID NO: 58 |
| 784 cl5 | SEQ ID NO: 71 | SEQ ID NO: 12 | SEQ ID NO: 58 |
| 784 cl10 | SEQ ID NO: 72 | SEQ ID NO: 12 | SEQ ID NO: 58 |
| 784 cl4 | SEQ ID NO: 73 | SEQ ID NO: 12 | SEQ ID NO: 58 |
| 784 cl8 | SEQ ID NO: 74 | SEQ ID NO: 12 | SEQ ID NO: 58 |
| 784 cl2 | SEQ ID NO: 75 | SEQ ID NO: 12 | SEQ ID NO: 58 |
| 784 cl3 | SEQ ID NO: 76 | SEQ ID NO: 12 | SEQ ID NO: 58 |
| 784 cl7 | SEQ ID NO: 77 | SEQ ID NO: 12 | SEQ ID NO: 58 |
| 784 cl6 | SEQ ID NO: 78 | SEQ ID NO: 12 | SEQ ID NO: 58 |
| 787 cl1 | SEQ ID NO: 79 | SEQ ID NO: 12 | SEQ ID NO: 59 |
| 787 cl7 | SEQ ID NO: 80 | SEQ ID NO: 12 | SEQ ID NO: 59 |
| 787 cl3 | SEQ ID NO: 81 | SEQ ID NO: 12 | SEQ ID NO: 59 |
| 787 cl5 | SEQ ID NO: 82 | SEQ ID NO: 12 | SEQ ID NO: 59 |
| 787 cl6 | SEQ ID NO: 83 | SEQ ID NO: 12 | SEQ ID NO: 59 |
| 787 cl10 | SEQ ID NO: 84 | SEQ ID NO: 12 | SEQ ID NO: 59 |
| 787 cl4 | SEQ ID NO: 85 | SEQ ID NO: 12 | SEQ ID NO: 59 |
| 787 cl8 | SEQ ID NO: 86 | SEQ ID NO: 12 | SEQ ID NO: 59 |
| 787 cl9 | SEQ ID NO: 87 | SEQ ID NO: 12 | SEQ ID NO: 59 |
| 787 cl2 | SEQ ID NO: 88 | SEQ ID NO: 12 | SEQ ID NO: 59 |

TABLE 5
Bxb1 LSR variants and their target sites.

| LSR | Amino acid | | Second target |
|---|---|---|---|
| variant | sequence | First target site (P) | site (B) |
| SH1 | SEQ ID NO: 8395 | SEQ ID NO: 8396 | SEQ ID NO: 8397 |
| cl10rep | | | |
| SH1 | SEQ ID NO: 8398 | SEQ ID NO: 8396 | SEQ ID NO: 8397 |
| cl4rep | | | |
| SH1 cl8 | SEQ ID NO: 8399 | SEQ ID NO: 8396 | SEQ ID NO: 8397 |
| SH1 cl3 | SEQ ID NO: 8400 | SEQ ID NO: 8396 | SEQ ID NO: 8397 |
| SH1 cl2 | SEQ ID NO: 8401 | SEQ ID NO: 8396 | SEQ ID NO: 8397 |
| SH1 cl9 | SEQ ID NO: 8402 | SEQ ID NO: 8396 | SEQ ID NO: 8397 |
| SH2 cl6 | SEQ ID NO: 8403 | SEQ ID NO: 8404 | SEQ ID NO: 8405 |
| SH2 cl7 | SEQ ID NO: 8406 | SEQ ID NO: 8404 | SEQ ID NO: 8405 |
| SH2 cl5 | SEQ ID NO: 8407 | SEQ ID NO: 8404 | SEQ ID NO: 8405 |
| SH2 cl8 | SEQ ID NO: 8408 | SEQ ID NO: 8404 | SEQ ID NO: 8405 |
| SH2 cl4 | SEQ ID NO: 8409 | SEQ ID NO: 8404 | SEQ ID NO: 8405 |
| SH2 cl3 | SEQ ID NO: 8410 | SEQ ID NO: 8404 | SEQ ID NO: 8405 |
| SH2 cl1 | SEQ ID NO: 8411 | SEQ ID NO: 8404 | SEQ ID NO: 8405 |
| SH3 cl4 | SEQ ID NO: 8412 | SEQ ID NO: 8413 | SEQ ID NO: 8414 |
| SH3 cl5 | SEQ ID NO: 8415 | SEQ ID NO: 8413 | SEQ ID NO: 8414 |
| SH3 cl7 | SEQ ID NO: 8416 | SEQ ID NO: 8413 | SEQ ID NO: 8414 |
| SH3 cl6 | SEQ ID NO: 8417 | SEQ ID NO: 8413 | SEQ ID NO: 8414 |
| SH3 cl2 | SEQ ID NO: 8418 | SEQ ID NO: 8413 | SEQ ID NO: 8414 |
| SH3 cl9 | SEQ ID NO: 8419 | SEQ ID NO: 8413 | SEQ ID NO: 8414 |
| SH3 cl3 | SEQ ID NO: 8420 | SEQ ID NO: 8413 | SEQ ID NO: 8414 |
| SH3 cl8 | SEQ ID NO: 8421 | SEQ ID NO: 8413 | SEQ ID NO: 8414 |
| SH4 cl4 | SEQ ID NO: 8422 | SEQ ID NO: 8423 | SEQ ID NO: 8424 |
| SH4 cl3 | SEQ ID NO: 8425 | SEQ ID NO: 8423 | SEQ ID NO: 8424 |
| SH4 | SEQ ID NO: 8426 | SEQ ID NO: 8423 | SEQ ID NO: 8424 |
| cl10 | | | |
| SH4 cl8 | SEQ ID NO: 8427 | SEQ ID NO: 8423 | SEQ ID NO: 8424 |
| SH4 cl1 | SEQ ID NO: 8428 | SEQ ID NO: 8423 | SEQ ID NO: 8424 |
| SH4 cl7 | SEQ ID NO: 8429 | SEQ ID NO: 8423 | SEQ ID NO: 8424 |

Further LSR variants of the present invention comprise or consist of an amino acid sequence as laid out in one of SEQ ID NOs: 89 to 8391 and 8430 to 13115, or a sequence at least 85% identical thereto.

Particularly preferred Bxb1 LSR variants comprise one or more, and preferably all of the mutations with respect to Bxb1 LSR wildtype (SEQ ID NO: 8392) selected from the group consisting of:
(a) Q70R, V74A, T84A, I87T, I87V, K138R, N141S, T166A, E171V, V175A, H189R, D193N, N194D, K214R, Q221R, E229K, I240T, E242G, E242V, T254I, D257G, E273G, L275Q, V283A, A315S, P322S, D355G, D355N, N381S, E418G, E434G, T453A, L488S, S490G;
(b) V74A, T84A, I87T, I87V, N141S, T166A, V175A, K214R, Q221R, E229K, I240T, E242V, T254I, L275Q, A315S, D355G, D355N, E434G;
(c) T84A, I87T, N141S, T166A, V175A, Q221R, E229K;
(d) D14E, E24A, Q27R, V74A, 187T, I87V, Q92R, H95L, H95R, N141I, N141S, A145T, A145V, H146R, T166A, V175A, H189R, H203Y, Q221R, Q226R, E229K, K236R, I240T, E242G, E242K, E242V, T254I, D257G, E273G, L275Q, V283A, K284R, A315T, A315V, F331S, K333E, A341V, A343T, A347V, E351K, D355G, D355N, K365R, A374S, V385M, E418G, G422S, E431G, E434G, F439L, G440R, T453A, V460I, V466A, D478E, Q480R, E483G, Q484R, L488S, S490G, V491A, V492A, H496R, T497A, M499T, S500P;
(e) V74A, I87T, I87V, H95R, N141I, N141S, A145V, H146R, T166A, H203Y, Q221R, Q226R, E229K, K236R, I240T, E242G, E242V, T254I, D257G, E273G, L275Q, V283A, A315V, K333E, A341V, A343T, D355G, D355N, E434G, F439L, G440R, D478E, Q484R, L488S, V492A, H496R;
(f) I87T, H95R, N141I, A145V, Q221R, Q226R, E229K, I240T, E242V, T254I, L275Q, V283A, A315V, K333E, A341V, D355N, E434G, V492A;
(g) D45N, V74A, T84A, I87T, I87V, K138R, T166A, V175A, H189R, N194D, P197S, L200W, Q221R, E229K, I240T, E242V, T254I, E273G, E281V, V283A, A341V, D355G, D355N, A360T, N381S, E424G, S428P, E431G, E434G, T453A, V466A, D478G, Q480R, L488S, S490G;
(h) V74A, T84A, 187T, I87V, T166A, V175A, Q221R, E229K, I240T, E273G, A341V, D355N, E424G, E434G;
(i) I87T, I87V, Q221R, E229K, A341V, E424G;
(j) D14E, E24A, Q27R, D45N, Q70R, V74A, T84A, I87T, I87V, Q92R, H95L, H95R, K138R, N141S, N141I, A145T, A145V, H146R, T166A, E171V, V175A, H189R, D193N, N194D, P197S, L200W, H203Y, K214R, Q221R, Q226R, E229K, K236R, I240T, E242G, E242V, E242K, T254I, D257G, E273G, L275Q, E281V, V283A, K284R, A315S, A315T, A315V, P322S, F331S, K333E, A341V, A343T, A347V, E351K, D355G, D355N, A360T, K365R, A374S, N381S, V385M, E418G, G422S, E424G, S428P, E431G, E434G, F439L, G440R, T453A, V460I, V466A, D478E, D478G, Q480R, E483G, Q484R, L488S, S490G, V491A, V492A, H496R, T497A, M499T, S500P;
(k) V74A, T84A, I87T, I87V, H95R, N141S, N141I, A145V, H146R, T166A, V175A, H203Y, K214R, Q221R, Q226R, E229K, K236R, I240T, E242V, E242G, T254I, D257G, E273G, L275Q, V283A, A315S, A315V, K333E, A341V, A343T, D355G, D355N, E424G, E434G, F439L, G440R, D478E, Q484R, L488S, V492A, H496R; or
(l) T84A, 187T, I87V, H95R, N141S, N141I, A145V, T166A, V175A, Q221R, Q226R, E229K, I240T, E242V, T254I, L275Q, V283A, A315V, K333E, A341V, D355N, E424G, E434G, V492A.

According to one embodiment of the present invention, the LSR is present as a monomer. According to a preferred embodiment of the present invention, the LSR comprises at least two LSR monomers, i.e. is in a dimeric form (dimer). Such a dimer can comprise two monomers of the same type (i.e. two identical integrase monomers, homodimer) or two monomers of a different type (i.e. two different integrase monomers, heterodimer). According to a further preferred embodiment of the present invention, the LSR comprises at least four protein monomers, i.e. is in a tetrameric form (tetramer). Such a tetramer can comprise four monomers of the same type (i.e. four identical integrase monomers, homotetramer), or monomers of a different type such as two, three or four different integrase monomers (heterotetramer). According to a particularly preferred embodiment, the variant LSR is present as a dimer and comprises two identical LSR monomers.

The present invention further pertains to a nucleic acid or group of nucleic acids encoding a variant LSR according to the present invention. For activation of the expression of such nucleic acid or group of nucleic acids, the nucleic acid encoding for the LSR preferably further comprises a regulatory nucleic acid sequence, preferably a promoter region. Hence, expression of the nucleic acid encoding for the integrase protein can be initiated or regulated by activating the regulatory nucleic acid sequence. Accordingly, one way for inducing a DNA integration of a donor nucleic acid into a cell or genome of a subject is introducing the nucleic acid sequence or group of nucleic acid sequences of the present invention into the respective subject or cell, and activating the regulatory nucleic acid sequence (preferably the promoter region) to express the gene encoding for the integrase protein. Preferably, the regulatory nucleic acid sequence (preferably the promoter region) is either introduced into a respective cell, preferably together with the sequence encoding for the integrase protein, or the regulatory nucleic acid sequence is already present in said cell at the beginning. In the second case, merely the nucleic acid encoding for the integrase protein is introduced into the cell (and placed under the control of the regulatory nucleic acid sequence).

The term “regulatory nucleic acid sequence” as used herein refers to gene regulatory regions of DNA. In addition to promoter regions, this term encompasses operator regions more distant from the gene as well as nucleic acid sequences that influence the expression of a gene, such as cis-elements, enhancers or silencers. The term “promoter region” as used herein refers to a nucleotide sequence on the DNA allowing a regulated expression of a gene. The promoter region allows regulated expression of the nucleic acid encoding for the respective protein. The promoter region is located at the 5′-end of the gene and thus before the RNA coding region. Both, bacterial and eukaryotic promoters are applicable for the invention.

According to one embodiment of the invention, the target sites are either already included in the subject or cell, or they are introduced into the subject's genome or into the cell, preferably by recombinant techniques well known to the person of ordinary skill in the art.

According to one preferred embodiment, the nucleic acid that is to be integrated (i.e. the donor nucleic acid) is already present in the subject and particularly in a respective cell. Alternatively, the nucleic acid to be integrated can be introduced into the subject or cell by conventional means known to the skilled person, such as by recombinant techniques (e.g. transfection or transduction).

The present invention also includes one or a plurality of nucleic acids or nucleic acid sequences or polynucleotides in which the coding sequence for the LSR is fused in the same reading frame to a polynucleotide sequence which aids in expression and secretion of a protein from a host cell. For example, a leader sequence, which functions as a secretory sequence for controlling transport of a polypeptide from the cell, may be fused to the sequence encoding the integrase protein. The polypeptide or protein having such a leader sequence is termed a pre-protein or a pre-proprotein and may have the leader sequence cleaved by the host cell to form the mature form of the protein. These polynucleotides may have a 5′ extended region so that it encodes a proprotein, which is the mature protein plus additional amino acid residues at the N-terminus. The expression product having such a pro-sequence is termed a pro-protein, which is an inactive form of the mature protein; however, once the pro-sequence is cleaved, an active mature protein remains. The additional sequence may also be attached to the protein and be part of the mature protein. Thus, for example, the polynucleotides of the present invention may encode polypeptides, or proteins having a pro-sequence, or proteins having both, a pro-sequence and a pre-sequence (such as a leader sequence).

The nucleic acids of the present invention may also have the coding sequence fused in frame to a marker sequence, which allows for purification of the integrase protein of the present invention. The marker sequence may be an affinity tag or an epitope tag such as a polyhistidine tag, a streptavidin tag, an Xpress tag, a FLAG tag, a cellulose or chitin binding tag, a glutathione-S transferase tag (GST), a hemagglutinin (HA) tag, a c-myc tag or a V5 tag.

If the nucleic acid of the invention is an mRNA, in particular for use as a medicament, the delivery of mRNA therapeutics can be facilitated by the significant progress that has been achieved in maximizing the translation and stability of mRNA, preventing its immune-stimulatory activity and the development of in vivo delivery technologies. The 5′ cap and 3′ poly(A) tail are the main contributors to efficient translation and prolonged half-life of mature eukaryotic mRNAs. Incorporation of cap analogs such as ARCA (anti-reverse cap analogs) and poly(A) tail of 120-150 bp into in vitro transcribed (IVT) mRNAs has markedly improved expression of the encoded proteins and mRNA stability. New types of cap analogs, such as 1,2-dithiodiphosphate-modified caps, with resistance against RNA decapping complex, can further improve the efficiency of RNA translation. Replacing rare codons within mRNA protein-coding sequences with synonymous frequently occurring codons, so-called codon optimization, also facilitates better efficacy of protein synthesis and limits mRNA destabilization by rare codons, thus preventing accelerated degradation of the transcript. Similarly, engineering 3′ and 5′ untranslated regions (UTRs), which contain sequences responsible for recruiting RNA-binding proteins (RBPs) and miRNAs, can enhance the level of protein product. Interestingly, UTRs can be deliberately modified to encode regulatory elements (e.g., K-turn motifs and miRNA binding sites), providing a means to control RNA expression in a cell-specific manner. Some RNA base modifications such as N1-methyl-pseudouridine have not only been instrumental in masking mRNA immune-stimulatory activity but have also been shown to increase mRNA translation by enhancing translation initiation. In addition to their observed effects on protein translation, base modifications and codon optimization affect the secondary structure of mRNA, which in turn influences its translation. Respective modifications of the nucleic acid molecules of the invention are also contemplated by the invention.

The RNA or plurality of RNAs preferably encode the integrase enzyme of the present invention or any of its subunits. Specific methods for delivering and expressing nucleic acids and specifically RNAs are disclosed e.g. in EP2590676 and EP3115064, which are herein incorporated by reference. The RNA may be present in a particle and is preferably self-replicating. After in vivo administration of the particles, RNA is released from the particles and is translated inside a cell to provide the DNA recombining enzyme or any of its monomeric subunits.

A self-replicating RNA molecule (replicon) can, when delivered to a vertebrate cell even without any proteins, lead to the production of multiple daughter RNAs by transcription from itself (via an antisense copy which it generates from itself). These daughter RNAs, as well as collinear sub-genomic transcripts, may be translated by themselves to provide in situ expression of an encoded polypeptide, or may be transcribed to provide further transcripts with the same sense as the delivered RNA which are translated to provide in situ expression of the polypeptide. The overall results of this sequence of transcriptions is a huge amplification in the number of the introduced replicon RNAs and so the encoded polypeptide becomes a major polypeptide product of the cells.

A preferred self-replicating RNA molecule encodes (i) an RNA-dependent RNA polymerase which can transcribe RNA from the self-replicating RNA molecule, and (ii) an integrase protein of the present invention. The polymerase can be an alphavirus replicase e.g. comprising one or more of alphavirus proteins nsP1, nsP2, nsP3 and nsP4. It is preferred that the self-replicating RNA molecules of the invention do not encode alphavirus structural proteins. Thus, a preferred self-replicating RNA can lead to the production of genomic RNA copies of itself in a cell, but not to the production of RNA-containing virions. A self-replicating RNA molecule useful in the context of the present invention may have two open reading frames. The first (5′) open reading frame encodes a replicase, and the second (3′) open reading frame encodes a polypeptide of the present invention. In some embodiments, the RNA may have additional (e.g. downstream) open reading frames e.g. for further encoding accessory polypeptides.

Such RNA is particularly suitable for the general use in gene therapy, and specifically for use in the treatment of genetic disorders or diseases.

The present invention further provides an expression vector comprising the nucleic acid according to the present invention. The expression vector is preferably a pEVO vector as described in Buchholz and Stewart, 2001, more preferably the variant pEVO vector described herein as pEVO4gg. According to a particularly preferred embodiment, the vector comprises a nucleic acid sequence encoding a protein having at least 85% identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115.

The present invention further provides a cell or host cell comprising the vector or the nucleic acid or group of nucleic acids according to the present invention. The skilled person readily identifies suitable host cells, which may be eukaryotic or prokaryotic. Preferred prokaryotic cells are bacterial cells. Particularly preferred prokaryotic cells are cells of Escherichia coli. Preferred eukaryotic cells are yeast cells (preferably Saccharomyces cerevisiae), insect cells, non-insect invertebrate cells, amphibian cells, or mammalian cells (preferably somatic or pluripotent stem cells, including embryonic stem cells and other pluripotent stem cells, like induced pluripotent stem cells, and other native cells or established cell lines, including NIH3T3, CHO, HeLa, HEK293, hiPS). According to a particularly preferred embodiment, the host cells are XL-1 Blue E. coli cells.

The present invention further provides a system for integrating a donor nucleic acid into a target nucleic acid such as into the genome of a subject or cell. The system comprises a polypeptide comprising a large serine recombinase (LSR) according to the present invention, or a nucleic acid encoding the same, and a donor nucleic acid to be inserted into the target nucleic acid. The donor nucleic acid preferably comprises at least one target site of the LSR.

The system according to the invention is applicable for use in combination with other recombinase systems and thus may become a particular valuable tool for genetic experiments where multiple recombinases are required simultaneously or sequentially.

The present invention also provides a pharmaceutical composition comprising the large serine recombinase according to the present invention and in particular a protein having at least 85% identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115, the one or more nucleic acids according to the present invention, or the vector according to the present invention, and optionally a pharmaceutically acceptable carrier. The pharmaceutical composition may be in any form that is suitable for the selected mode of administration.

In one embodiment, the pharmaceutical composition of the present invention is administered parenterally.

The phrases “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

The therapeutically active agents in the pharmaceutical composition of the invention include but are not limited to the large serine recombinase according to the present invention and include in particular a protein having at least 85% identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115, the one or more nucleic acids according to the present invention, or the vector according to the present invention. According to a preferred embodiment, the active agent in the pharmaceutical composition is the large serine recombinase according to the present invention and in particular a protein having at least 85% identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115. The pharmaceutical composition comprising the therapeutically active agent can be administered, as sole pharmaceutical composition, or in combination with other active agents, in a unit administration form, as a mixture with conventional pharmaceutical supports, to animals and human beings.

In further embodiments, the pharmaceutical composition contains one or more carriers (also termed vehicles) which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and storage and must be preserved against the contaminating action of microorganisms, such as bacteria and fungi.

According to a further aspect, the present invention provides the use of a large serine recombinase according to the present invention and in particular the use of a protein having at least 85% identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115 for integrating a donor nucleic acid into a target nucleic acid such as into the genome of a subject or cell. According to a particularly preferred embodiment, the donor nucleic acid encodes a polypeptide or protein of interest and is integrated into the target nucleic acid such that the polypeptide or protein of interest is expressed when the target nucleic acid is transcribed and translated. The donor nucleic acid preferably comprises at least one target site of the LSR.

According to a further aspect, the present invention provides the use of the nucleic acid or group of nucleic acids of the present invention for integrating a donor nucleic acid into a target nucleic acid such as into the genome of a subject or cell. According to a particularly preferred embodiment, the donor nucleic acid encodes a polypeptide or protein of interest and is integrated into the target nucleic acid such that the polypeptide or protein of interest is expressed when the target nucleic acid is transcribed and translated. The donor nucleic acid preferably comprises at least one target site of the LSR.

According to a further aspect, the present invention provides the use of the expression vector or the pharmaceutical composition of the present invention for integrating a donor nucleic acid into a target nucleic acid such as into the genome of a subject or cell. According to a particularly preferred embodiment, the donor nucleic acid encodes a polypeptide or protein of interest and is integrated into the target nucleic acid such that the polypeptide or protein of interest is expressed when the target nucleic acid is transcribed and translated. The donor nucleic acid preferably comprises at least one target site of the LSR.

According to a further aspect, the present invention provides the use of the system of the present invention for integrating a donor nucleic acid into a target nucleic acid such as into the genome of a subject or cell. According to a particularly preferred embodiment, the donor nucleic acid encodes a polypeptide or protein of interest and is integrated into the target nucleic acid such that the polypeptide or protein of interest is expressed when the target nucleic acid is transcribed and translated. The donor nucleic acid preferably comprises at least one target site of the LSR.

The present invention further provides the large serine recombinase according to the present invention and in particular a protein having at least 85% identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115, the nucleic acid or group of nucleic acids of the present invention, the expression vector of the present invention, the pharmaceutical composition of the present invention, or the system of the present invention for use in medicine.

The present invention further provides the large serine recombinase according to the present invention and in particular a protein having at least 85% identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115, the nucleic acid or group of nucleic acids of the present invention, the expression vector of the present invention, the pharmaceutical composition of the present invention, or the system of the present invention for use in the treatment of a genetic disease or disorder. The genetic disease or disorder is not particularly limited and includes any genetic disease or disorder that can be treated by integrating a nucleic acid sequence or plurality of nucleic acid sequences into the genome of a respective patient that encodes one or more proteins or polypeptides that aid in treating the genetic disease or disorder. A preferred genetic disease or disorder is a monogenetic disease or disorder.

According to one embodiment, a (host) cell within the meaning of the invention is preferably a naturally occurring cell or a cell line (optionally transformed or genetically modified) that comprises at least one vector according to the invention or a nucleic acid according to the invention recombinantly, as described above. Thereby, the invention includes transient transfectants (e.g. by mRNA injection) or host cells that include at least one expression vector according to the invention as a plasmid or artificial chromosome, as well as host cells in which an expression vector according to the invention is stably integrated into the genome of said host cell.

Further provided are kits comprising a therapeutically active agent as described herein. In one embodiment, the kit provides the therapeutically active agents prepared in one or more unitary dosage forms ready for administration to a subject, for example in a preloaded syringe or in an ampoule. In another embodiment, the therapeutically active agents are provided in a lyophilized form.

Using the present invention, it is possible to integrate a donor nucleic acid, preferably DNA, into a target nucleic acid, preferably DNA, which target nucleic acid can be present in a host organism, such as mammals. Therefore, the present invention also includes a host organism comprising the integrated nucleic acid. According to one embodiment, the host organism is not a human. Also provided are host organisms which comprise a vector according to the invention or a nucleic acid according to the invention as described above that is, respectively, stably integrated into the genome of the host organism or individual cells of the host organism. Preferred host organisms according to the present invention are plants, invertebrates and vertebrates, particularly Bovidae, Drosophila melanogaster, Caenorhabditis elegans, Xenopus laevis, medaka, zebrafish, or Mus musculus, or embryos of these organisms.

The present invention further pertains to a method for integrating a donor DNA into a target nucleic acid such as into the genome of a subject or cell. The method comprises the steps of (i) providing the LSR variant according to the present invention and in particular a protein comprising or consisting of an amino acid sequence having at least 85% identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115, the nucleic acid or group of nucleic acids of the present invention, the expression vector of the present invention, the pharmaceutical composition of the present invention, or the system of the present invention, and a donor DNA, optionally allowing the large serine recombinase to be expressed, and (ii) allowing the LSR to integrate the donor nucleic acid into the target nucleic acid.

The present invention further pertains to a method for treating a genetic disease or disorder in a subject by integrating a donor DNA into a target nucleic acid of the subject. The method comprises the steps of (i) providing the LSR variant according to the present invention and in particular a protein comprising or consisting of an amino acid having at least 85% identity to any one of SEQ ID NOs: 14, 17 to 35, 38 to 56, 60 to 8391, 8395, 8398 to 8403, 8406 to 8412, 8415 to 8422, 8425 to 8429, 8430 to 13115, the nucleic acid or group of nucleic acids of the present invention, the expression vector of the present invention, the pharmaceutical composition of the present invention, or the system of the present invention, and a donor DNA, optionally allowing the large serine recombinase to be expressed, and (ii) allowing the large serine recombinase to integrate the donor nucleic acid into the target nucleic acid.

The present invention further pertains to the following items.

Item 1: A method for generating a target-specific large serine recombinase (LSR), the method comprising the steps of:
a) generating a plurality of first variants of an LSR by introducing one or more mutations into the sequence encoding an LSR;
b) generating a library of expression vectors, wherein each expression vector comprises a first region comprising the sequence encoding one of the first variants of the LSR, and a second region comprising at least two first target sites of the LSR, wherein the two first target sites are different from each other;
c) introducing the library of expression vectors into host cells;
d) culturing the host cells and expressing the first variant LSR;
e) isolating plasmid DNA from the culture of host cells;
f) determining whether the portion of the second region on the expression vector between the two first target sites has been excised by the first variant LSR;
g) repeating step a) with those first LSR variants that excised the DNA sequence of the second region on the expression vector between the two first target sites by introducing one or more mutations into the sequence encoding the first variant LSR to generate second variants of the LSR;
h) repeating steps b) to f) optionally with at least one second target site, wherein the at least one second target site differs from the first target sites in at least one nucleotide.

Item 2: The method according to item 1, further comprising the step of repeating steps g) and h), preferably wherein steps g) and h) are repeated at least twice to at least 30 times.

Item 3: The method according to item 1 or 2, wherein the LSR in step a) as basis for the plurality of first variants of the LSR is a naturally occurring LSR.

Item 4: The method according to any one of items 1 to 3, wherein the two target sites in the second region of the expression vector are spaced apart by at least 50 nucleotides.

Item 5: The method according to any one of items 1 to 4, wherein the determining step f) comprises (i) sequencing of the first and the second region of the expression vector, or (ii) performing restriction digestion on the sequence of the expression vector comprising the two target sites followed by analysis of the digestion fragments.

Item 6: The method according to any one of items 1 to 5, further comprising the step of removing inactive variants of the variant LSR from the library of expression vectors.

Item 7: The method according to any one of items 1 to 6, wherein the one or more mutations in the sequence encoding the LSR are introduced by random mutagenesis using error-prone PCR.

Item 8: The method according to any one of items 1 to 7, wherein the first region on the vector further comprises a unique molecular identifier.

Item 9: The method according to item 8, further comprising one or more of the steps of:
(i) clustering the unique molecular identifiers,
(ii) generating and polishing consensus sequences,
(iii) determining the number of DNA modification events for each variant LSR;
(iv) determining an activity rate for each LSR variant; and/or
(v) selecting a target-specific LSR from the variant LSRs generated in step a) or steps g) to i) of claim 1 that excises the DNA sequence on the second region on the expression vector between two variant target sites of interest.

Item 10: The method according to any one of the preceding items, wherein the expression vector is a pEVO vector.

Item 11: A variant large serine recombinase (LSR) obtainable by the method according to any of items 1 to 10, wherein the amino acid sequence of the variant LSR differs in at least one amino acid from the amino acid sequence of the LSR from which the variant is generated in step a), and from the amino acid sequence of any other naturally occurring serine recombinase, preferably wherein the variant comprises or consists of an amino acid sequence having at least 85% identity to one of SEQ ID NOs: 14, 17 to 35, 38 to 56, 60 to 8391, 8395, 8398 to 8403, 8406 to 8412, 8415 to 8422, 8425 to 8429, and 8430 to 13115.

Item 12: The variant LSR according to item 11, wherein the variant LSR is a variant of an LSR selected from the group consisting of A118, TP901, φRV1, Bxb1, φC31, R4, Wβ, Tnpx, Cp36, Dn29, Kp03, Nm60, Pa01, Si74.

Item 13: A nucleic acid or group of nucleic acids encoding a variant LSR according to item 11 or 12.

Item 14: An expression vector comprising a nucleic acid or group of nucleic acids according to item 13.

Item 15: A system for integrating a donor DNA into a target nucleic acid, the system comprising
a polypeptide comprising a variant LSR according to item 11 or 12, or a nucleic acid encoding the same, and
a donor nucleic acid to be inserted into the target nucleic acid.

Item 16: A pharmaceutical composition comprising the variant LSR of item 11, the nucleic acid or group of nucleic acids according to item 13, or the expression vector according to item 14, and optionally a pharmaceutically acceptable carrier.

Item 17: Use of the variant LSR of item 11 or 12, the nucleic acid or group of nucleic acids of item 13, the expression vector of item 14, the system of item 15, or the pharmaceutical composition of item 16, for integrating a nucleic acid sequence of interest into the genome of a subject or cell, wherein the cell does not include cells of the human germ line.

Item 18: The variant LSR according to item 11 or 12, the nucleic acid or group of nucleic acids of item 13, the expression vector of item 14, or the pharmaceutical composition of item 16 for use in medicine.

Item 19: The variant LSR, the nucleic acid or group of nucleic acids, the expression vector, or the pharmaceutical composition for use of item 18, for use in the treatment of a genetic disease or disorder, preferably wherein the genetic disease or disorder is a monogenetic disease or disorder.

Item 20: A method for integrating a donor DNA into a target nucleic acid such as into the genome of a subject or cell, comprising the steps of (i) providing the LSR variant according to the present invention and in particular a protein comprising or consisting of an amino acid sequence having at least 85% identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115, the nucleic acid or group of nucleic acids of the present invention, the expression vector of the present invention, the pharmaceutical composition of the present invention, or the system of the present invention, and a donor DNA, optionally allowing the large serine recombinase to be expressed, and (ii) allowing the LSR to integrate the donor nucleic acid into the target nucleic acid.

Item 21: A method for treating a genetic disease or disorder in a subject by integrating a donor DNA into a target nucleic acid of the subject, the method comprising the steps of (i) providing the LSR variant according to the present invention and in particular a protein comprising or consisting of an amino acid having at least 85% identity to any one of SEQ ID NOs: 14, 17 to 35, 38 to 56, 60 to 8391, 8395, 8398 to 8403, 8406 to 8412, 8415 to 8422, 8425 to 8429, and 8430 to 13115, the nucleic acid or group of nucleic acids of the present invention, the expression vector of the present invention, the pharmaceutical composition of the present invention, or the system of the present invention, and a donor DNA, optionally allowing the large serine recombinase to be expressed, and (ii) allowing the large serine recombinase to integrate the donor nucleic acid into the target nucleic acid.

### EXAMPLES

### Example 1: Cloning of Integrase Genes

After analyzing the published literature and sequences available in GenBank, four large serine recombinases (A118, PhiRv1, TP901-1 and Bxb1) were selected and evolved as detailed herein. The amino acid sequences for large site-specific recombinases of the serine family were obtained from GenBank and reverse translated to DNA. Since the sources of recombinases were from various bacteria or bacterial viruses, the DNA sequence was optimized for recombinase expression in E. Coli without changing the encoded amino acid sequence. The genes were synthesized with restriction enzyme sites for cloning. A 20 μl digest reaction was prepared as follows: 500 ng plasmid DNA, 2 μl CutSmart Buffer, 1 μl XbaI, 1 μl BsrGI-HF, and water. The reaction was incubated at 37° C. for at least 1 h and the insert was purified using ISOLATE II PCR and Gel Kit (Bioline) according to the manufacturer's instructions. The purified insert was subsequently used for ligation with the pEVO4gg vector containing target sites. The pEVO4gg vector was made based on the pEVO4 vector by integrating oligo with BbsI target sites (Hoersten et al., unpublished). A map of the pEVO4gg vector is shown in FIG. 7.

### Example 2: Target Site Cloning

To generate a plasmid with target sites (see Tables 2 to 5), oligonucleotides containing the target sites were used to amplify a DNA fragment that was cloned into a pEVO4gg backbone via Golden Gate. This method offers a proficient and directional means for the concurrent assembly of multiple DNA fragments into a unified construct, employing Type IIS restriction enzymes and T4 DNA ligase as key components of the process. Type IIS enzymes like BbsI cut DNA outside of their recognition sites and because the final product lacks the recognition site, the correctly ligated product cannot be cut again by the restriction enzyme, meaning the reaction is essentially irreversible (Engler et al., 2008). Specifically, a 50 μl PCR reaction was prepared as follows: 36 μl water, 10 μl 5× Herculase II Reaction Buffer, 0.5 μl 100 mM dNTP Mix, 1.25 μl of each forward and reverse primer (10 μM), 0.5 μl Herculase II Fusion DNA Polymerase, 1 μl of any pEVO4 or pEVO4gg vector with target sites (20 ng/μl). The reaction was run with the following program: 95° C. 3 min; 30 cycles: 95° C. 15 s, 55° C. 20 s, 72° C. 30 s; 72° C. 3 min; store at 8° C. PCR product was directly used for Golden Gate reaction.

TABLE 6
Primers used in the excision evolution assay.

| Primer | Primer sequence |
|---|---|
| F | TTTGAAGACCCCAC |
| | (SEQ ID NO: 1) |
| | -target_site_1- |
| | AGCTTGCATGCCTGCAGATCGAG |
| | (SEQ ID NO: 2) |
| R | TTTGAAGACCTAAAC |
| | (SEQ ID NO: 3) |
| | -rev_comp_target_site_2- |
| | tcgaactgtaccggttgttagtga |
| | (SEQ ID NO: 4) |

A 20 μl Golden Gate reaction was prepared as follows: 2 μl Fast Digest Buffer, 2 μl 10 mM ATP, 75 ng of pEVO4gg vector, 1 μl PCR product with target sites (diluted 1:10), 1 μl T4 DNA Ligase, 1 μl BpiI-HF, and water. The reaction was run with the following program: 37° C. for 10 minutes, 10 cycles of 37° C. for 5 minutes and 16° C. for 5 minutes, 37° C. for 15 minutes, and 65° C. for 10 minutes.

Golden Gate reaction was then used for bacterial transformation. The entire transformation mixture was plated on agar plates containing 15 μg/ml Chloramphenicol, and the cultures were grown in an incubator at 37° C. for 14-16 hours.

### Example 3: Excision Evolution Assay

The integrase gene was amplified with error-prone PCR to create a library. PCRs were performed with a low-fidelity DNA polymerase (MyTaq, Bioline) and PCR products were digested with XbaI and BsrGI for further cloning. Digested fragments were isolated from an agarose gel with the Isolate II PCR and Gel Kit (Bioline) and ligated into similarly digested pEVO4gg vector with target sites. Ligation was performed between 30 ng insert and 60 ng vector using T4 DNA Ligase (NEB).

Ligated plasmids were desalted with MF-Millipore membrane filters (Merck) on distilled water for 20 minutes and transformed into XL-1 Blue E. coli cells via electroporation. Transformed bacteria were cultured in SOC medium for 1 h at 37° C. 2 μl of this culture were spread on agarose plates with 15 mg/ml chloramphenicol and incubated over night at 37° C. The number of colonies on the plates was determined to calculate the number of transformed bacteria present per μl of SOC culture. The rest of the transformation solution was added to 100 ml LB medium to grow the library overnight at 37° C. Liquid cultures contained a specific amount of arabinose to induce the expression of the integrase. Arabinose levels and thus induction of enzyme expression used in the evolution were 200 μg/ml, 100 μg/ml, 50 μg/ml, 25 μg/ml, 10 μg/ml, 5 μg/ml and 1 μg/ml. Arabinose solution was prepared from the powder to the stock concentration of 100 mg/ml and then diluted with LB-medium to reach required concentration.

Plasmid DNA of these cultures was subsequently extracted using the GeneJet Plasmid Miniprep Kit (Thermo Fisher Scientific) and was used to characterize excision and continue evolution cycles. To continue the evolution cycle, a miniprep of plasmid DNA was digested with NdeI and AvrII, which target the plasmid portion in between target sites on the vector. In the absence of an excision reaction, the vector is linearized and primers for PCR amplification will go in opposite directions. If an excision reaction took place, restriction sites are removed and the vector stays intact, preserving correct orientation the PCR primers.

A further round of directed evolution was started with an error-prone-PCR on the enzymatically digested DNA. Only edited plasmids were replicated because primers are placed in a way that only the edited DNA fragment is a valid template for PCR. The PCR product was subsequently cloned into non-edited pEVO4gg vectors, which started a new evolution cycle. For increasing selection pressure, arabinose levels and thus induction of enzyme expression were lowered from 200 μg/ml to 1 μg/ml.

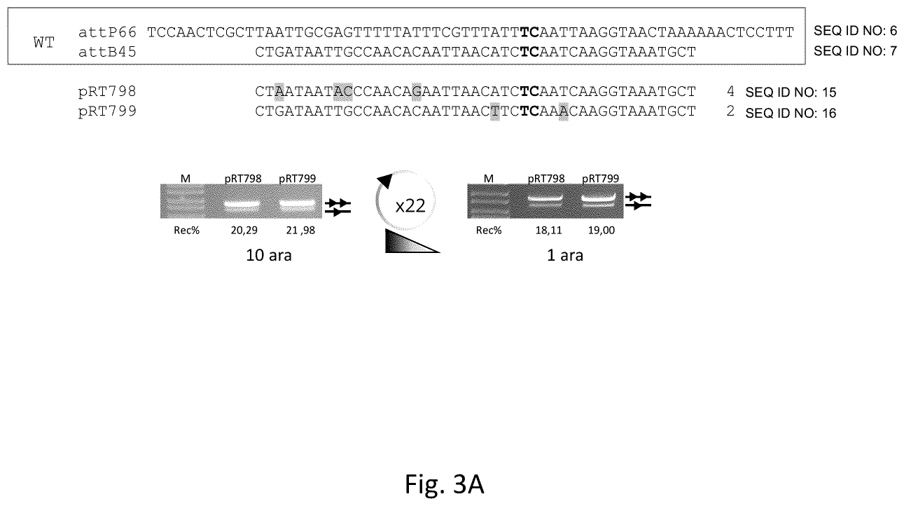

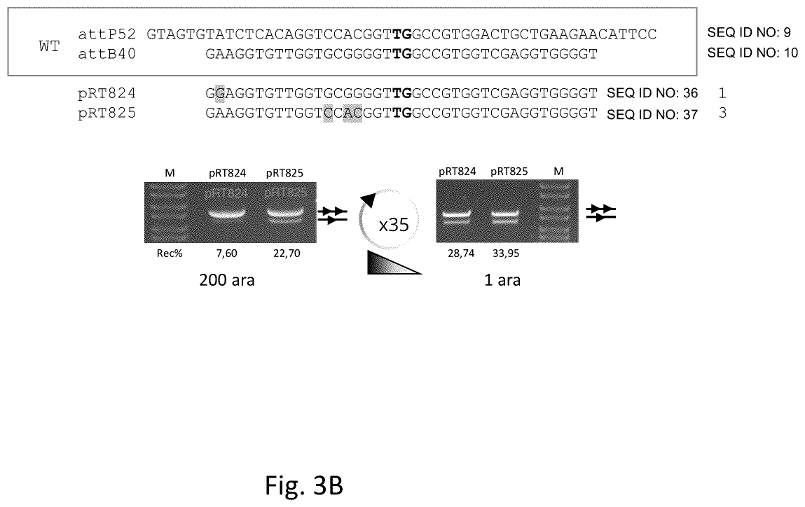

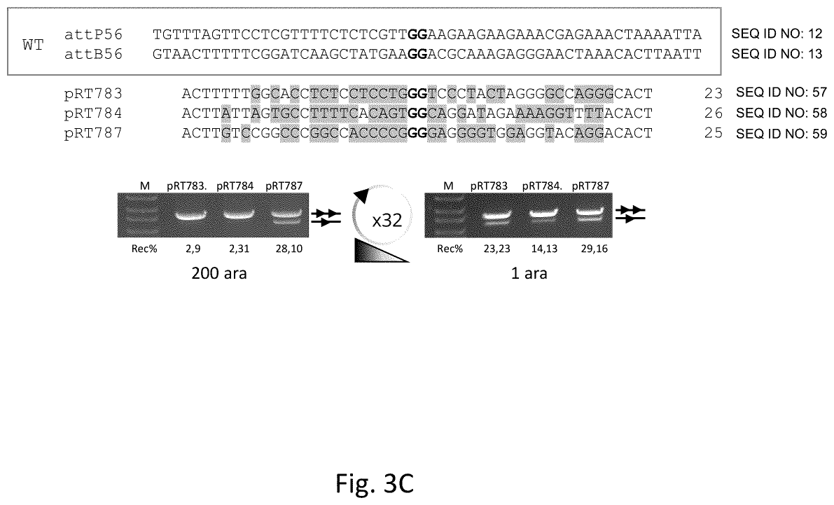

The evolution processes performed on the integrases TP901, PhiRv1, and A118 are illustrated in FIGS. 3A to 3C, respectively; Tables 2 to 4 summarize the respective sequences of the variant LSRs and the target sites on which the variants have been evolved. In FIGS. 3A to 3C, sequences of the wild-type target sites are presented in the top panel termed WT. New target sites used for evolution are shown below said box. Grey squares within the new target site sequences mark changes in the new target sites. Total number of changes is given at the end of each new target sequence. Agarose gel pictures in the lower panel show the results of the recombination tests and respective activities in percent (Rec %). For determining activity, recombined and non-recombined plasmids are digested with restriction enzymes that excise the recombinase gene. Due to differences in plasmid sizes between the two versions, the resulting DNA fragments containing the plasmid backbone are also different in size. The size difference is visualized in agarose gel electrophoresis. Because enzymes in the evolution library have different efficiencies, results are a mixture of recombined and non-recombined plasmids. When an agarose gel picture contains both kinds of fragments, the strength of each band corresponds to the amount of the respective fragment, allowing quantification of the recombination rate. Left agarose pictures represent the start of the evolution process, the right agarose pictures represent the end of the evolution process. Pictograms at the right of the gel scheme indicate to which fragment each band corresponds to: two triangles represent non-recombined fragments, and one triangle represents recombined fragments. The total number of evolution cycles is shown in the circle arrow in the middle between both agarose gel pictures. Arabinose induction level in μg/ml is noted under the gel pictures; M denotes the molecular weight marker.

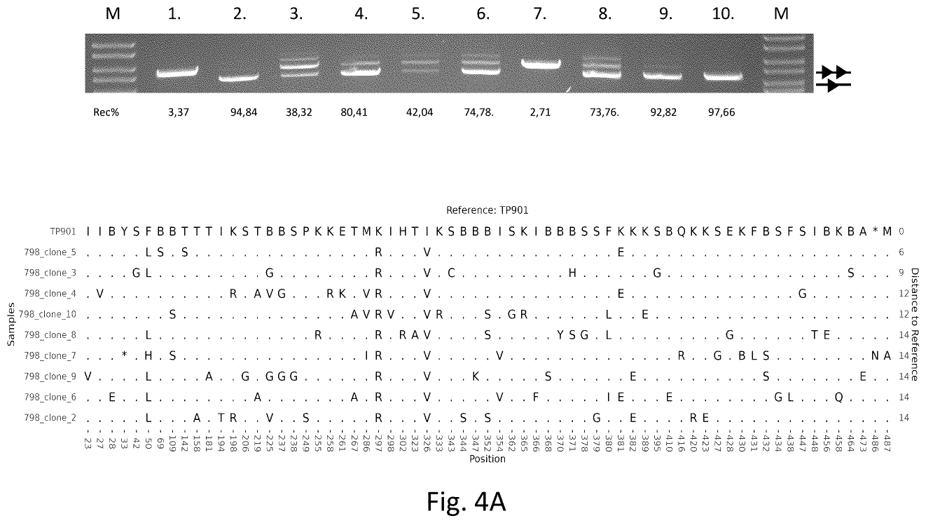

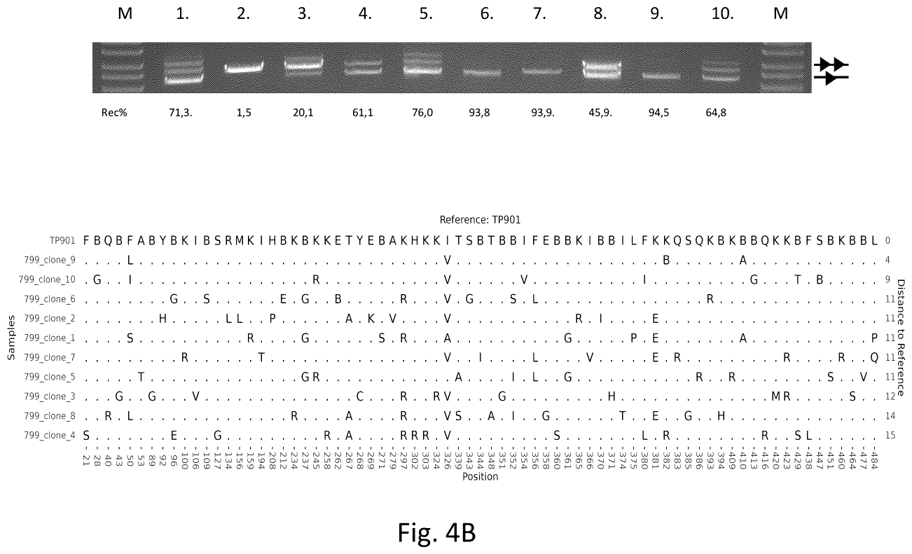

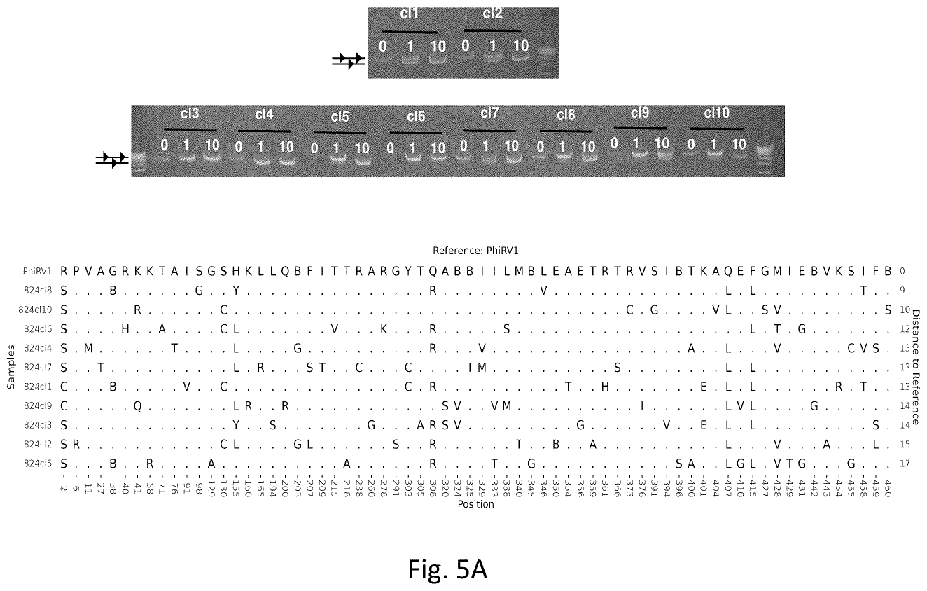

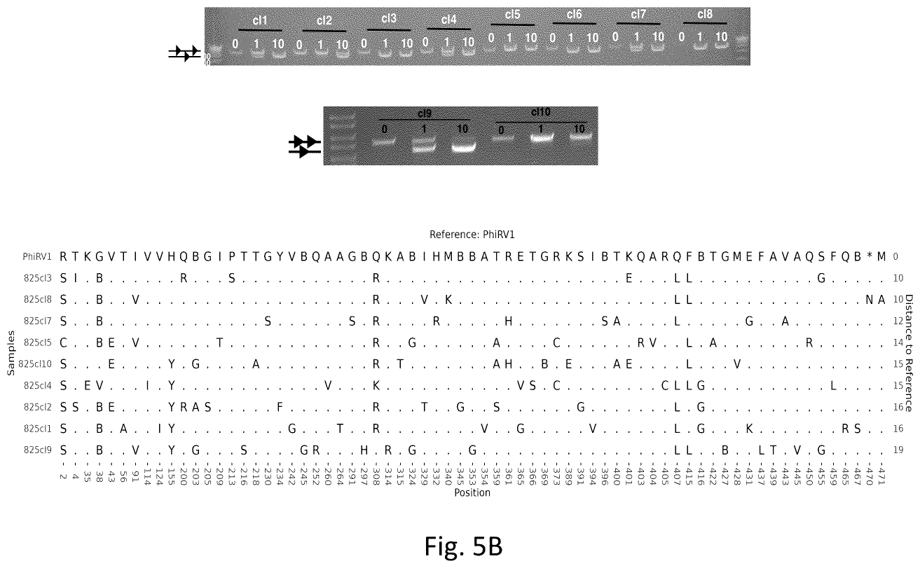

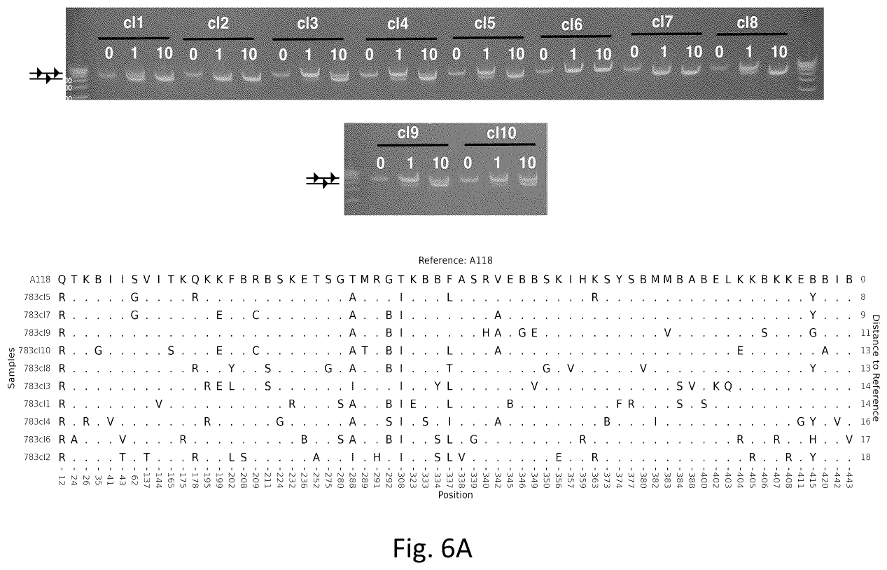

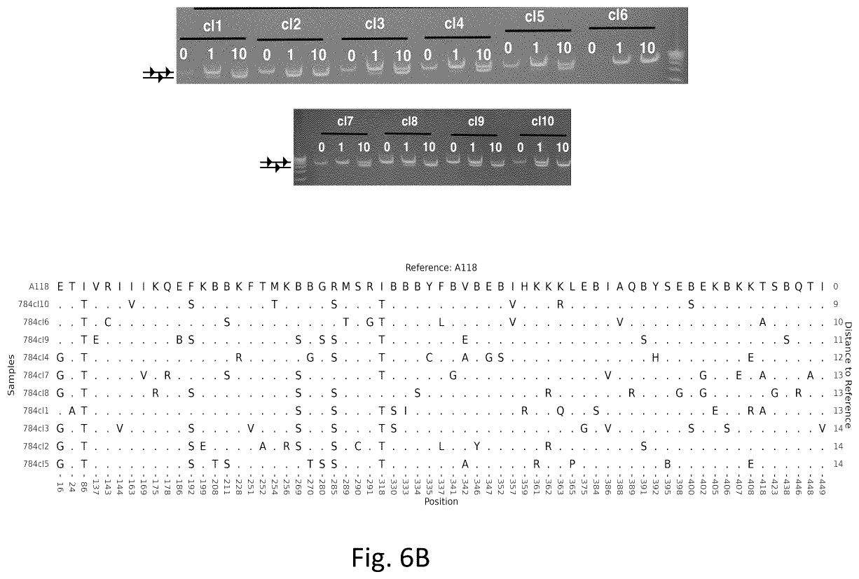

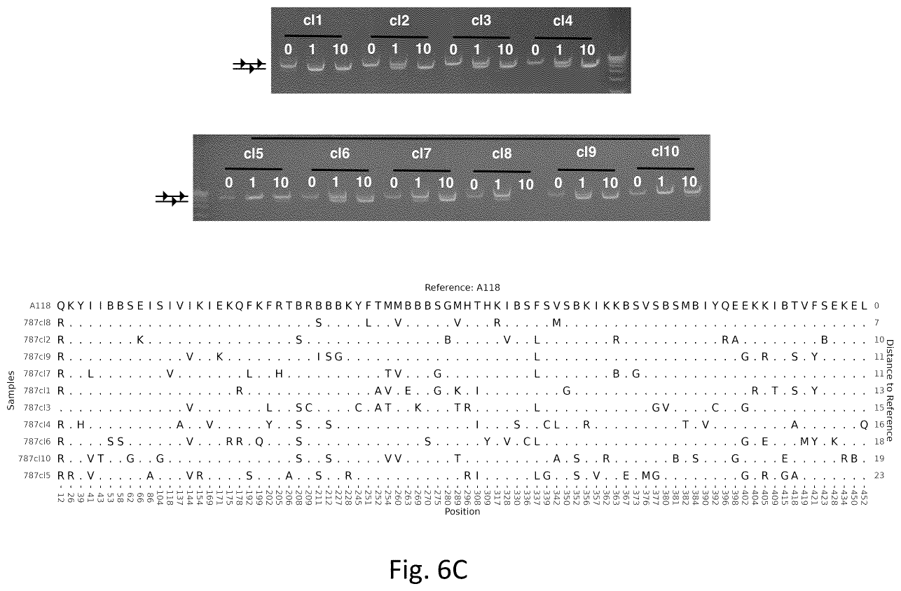

Resulting integrase variants are shown in FIG. 4 (TP901 variants), FIG. 5 (PhiRv1 variants), and FIG. 6 (A118 variants). For each library, ten clones were analyzed under arabinose induction as indicated (1 μg/ml for TP901 variants (FIG. 4), and 0, 1 and 10 μg/ml for the other variants (FIGS. 5 to 7)). Every clone was sequenced with Sanger sequencing using forward and reverse primers. Two reads were used to create one gene sequence, translated und used for amino acid sequence alignment compared to the wild-type enzyme. The sequence alignment of each clone with the wild-type enzyme is shown in each figure. No full sequence alignments are shown but the figures highlight only those positions that differ between the variants and the wildtype sequence. A dot represents no change, * is a stop codon, # is an insertion. Positions of each mismatch are indicated in the bottom line below each sequence alignment. Only positions with differences to the wildtype reference are shown. Sequence of the wild-type enzyme was used as a reference.

### Example 4: Test Digests

In order to detect whether an integrase variant catalyzed an excision reaction, a restriction digest was performed as shown in FIGS. 3 to 7. Integrases were cloned into pEVO vectors with the respective target sites by utilizing XbaI and BsrGI restriction enzymes. Expression of integrases was controlled by an L-arabinose inducible promoter system (araBAD). Recombination of the respective target sites on the evolution plasmid leads to the excision of a 750 bp fragment from the plasmid. The resulting size difference is mediated by the recombinase activity and is detectable by a restriction digest followed by gel electrophoresis. The size of leftover backbone correlates to the unmodified vector (5147 bp) or to a vector containing an excision (4397 bp) in proportion to the relative abundance of each.

### Example 5: Excision Evolution Assay

A transient fluorescence-based reporter assay described in Lansing et al. 2020, and in Lansing et al. 2022 was used to determine the recombination properties of the evolved integrase variants as.

Expi293F cells were maintained in Dulbecco's modified Eagle medium (Invitrogen GmbH) supplemented with 10% fetal bovine serum and 1% penicillin-streptomycin (10,000 U/ml, Thermo Fisher). Cells were passaged every 3 to 4 days and maintained at 37° C. and 5% CO₂. Cells were transfected using Lipofectamine 2000 (Invitrogen) according to manufacturer's instructions. After 48 hours, cells were harvested and analysed in a MACS Quant analyser (Miltenyi Biotec).

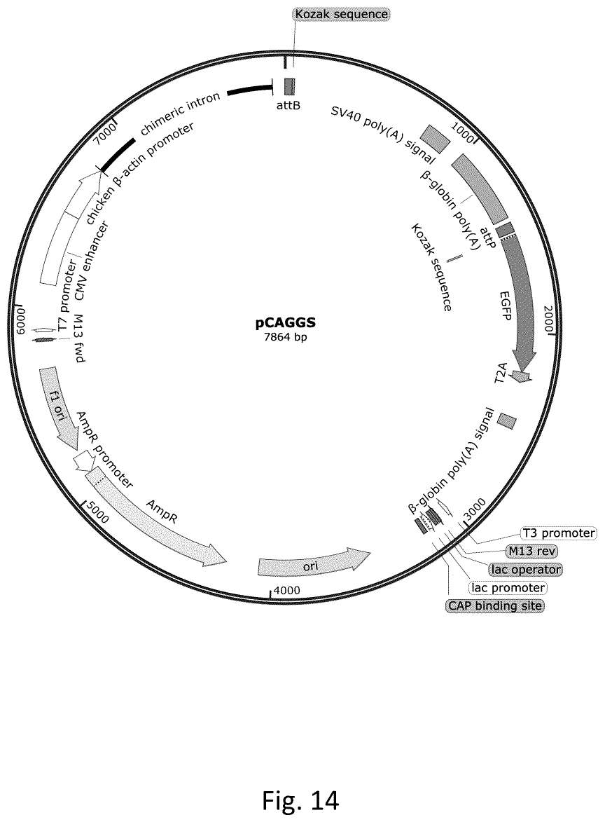

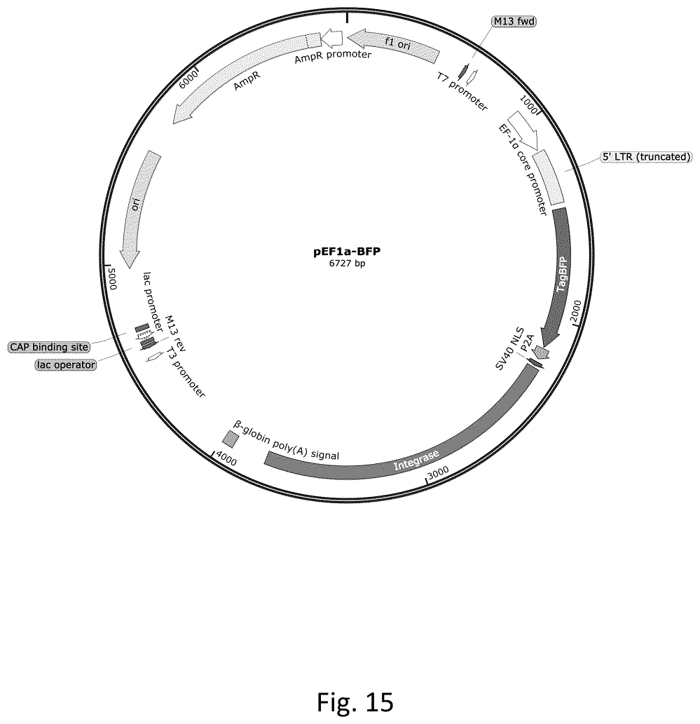

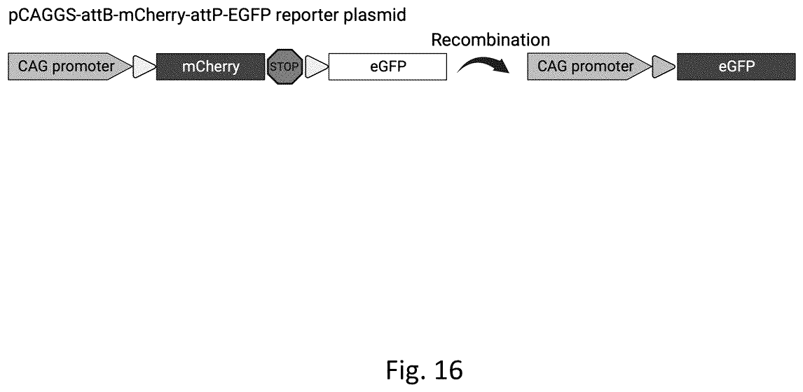

The test was performed as follows: 75,000 cells per well were seeded in a 24-well plate. Cells were co-transfected with combination of EF1a-tagBFP-P2A-Int plasmid (FIG. 15), encoding for one integrase variant and blue fluorescent protein (BFP), along with pCAGGS-attB-mCherry-attP-EGFP reporter plasmid (FIG. 14) encoding a red fluorescent protein mCherry flanked by the tested target sites. Upon recombination of these sites, a downstream EGFP gene is expressed. Schematic representation of this process is shown in FIG. 16. 48 hours after transfection, the recombinase activity was quantified via FACS using the MACSQuant VYB flow cytometer (Miltenyi Biotec). Analysis of the data was performed using FlowJo™ 10 (BD). Cell populations were grouped with forward scatter and side scatter to group the total cell population. Cells expressing BFP were grouped with forward scatter and BFP to define populations which had acquired EF1a-tagBFP-P2A-Int plasmid with the integrase variant. From the BFP population, cells were grouped by GFP and mCherry expression. Recombination was quantified by determining the percentage of cells displaying green fluorescence within the blue fluorescence population.

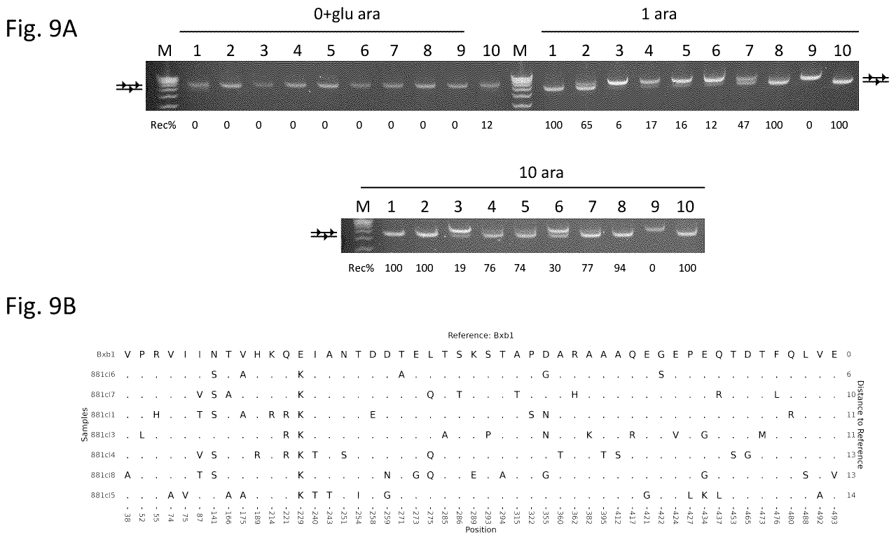

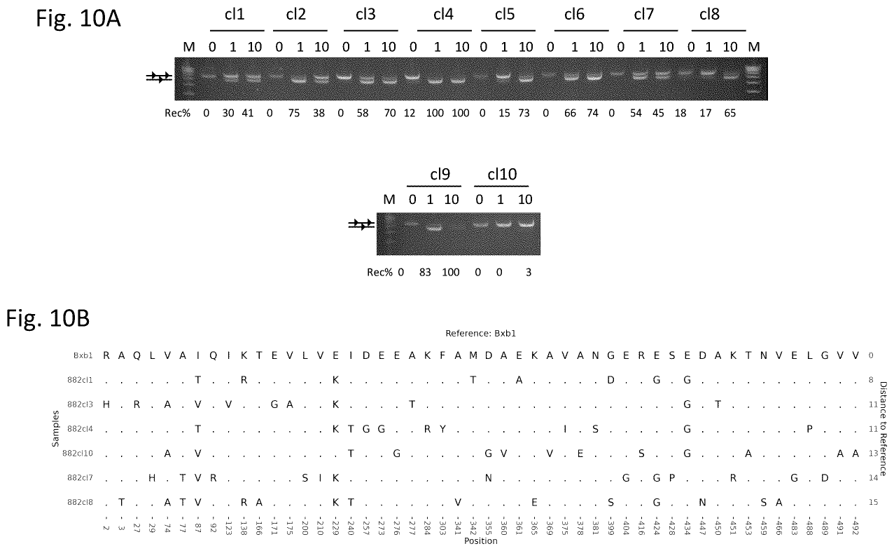

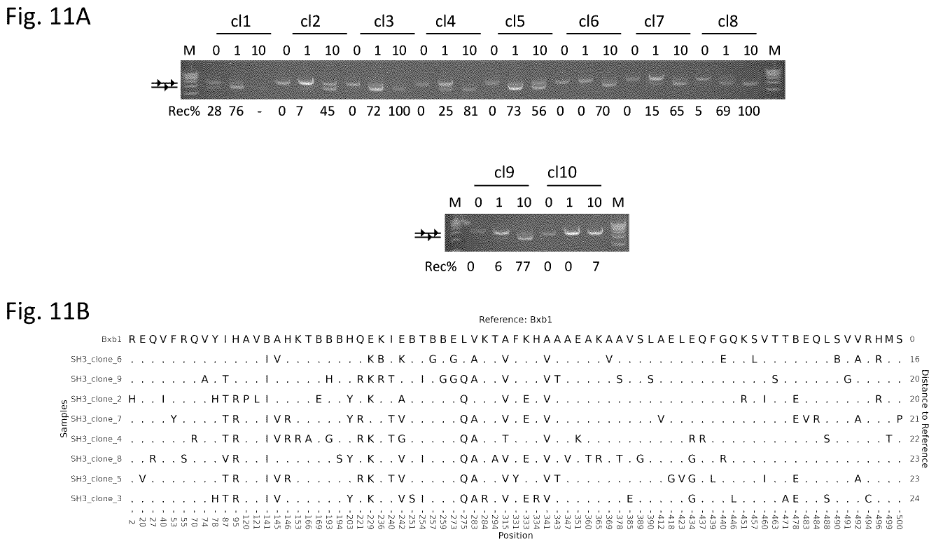

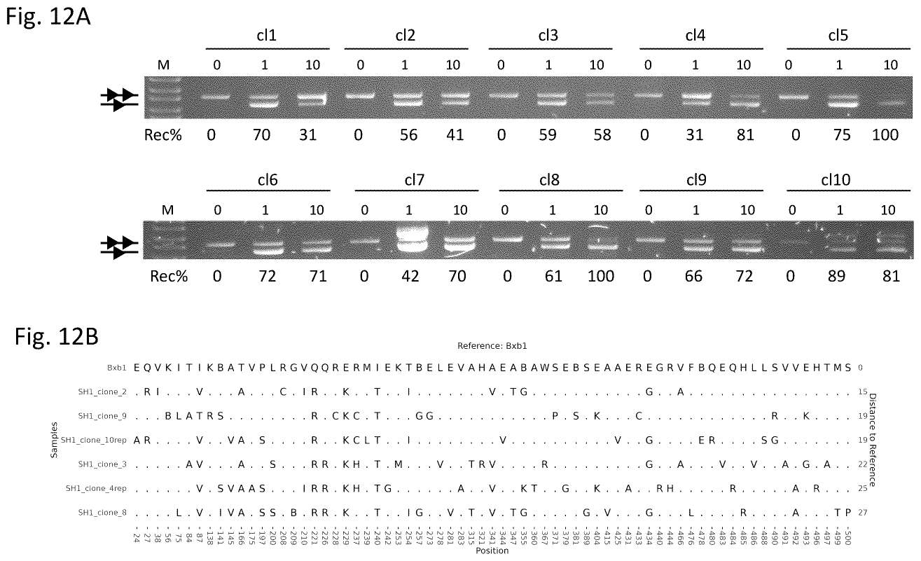

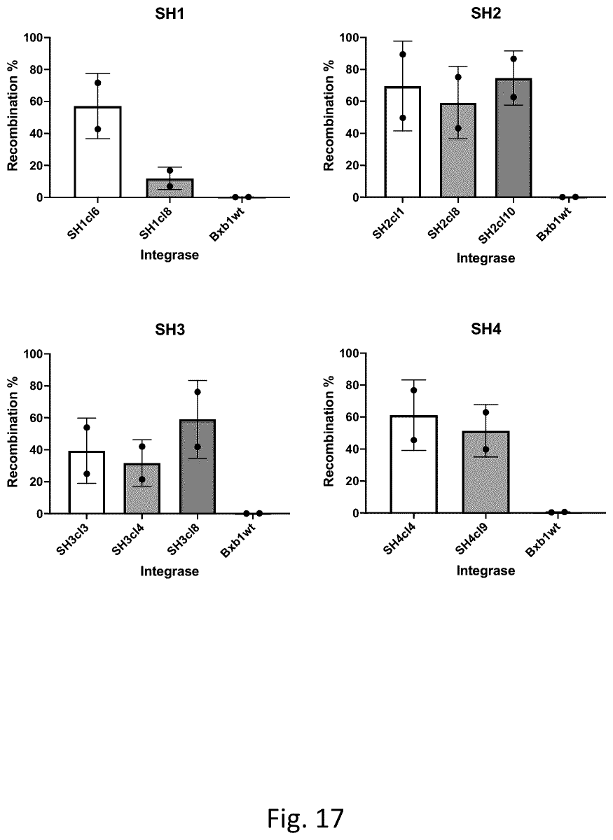

Two sets of Bxb1 variants were tested for recombination efficiency in mammalian cell culture. First, 2 to 3 variants per safe harbour side (SHS) library with the highest recombination efficiency from the tested single clones (FIG. 9 to 14) were assessed. Results are shown in FIG. 17. Wild-type Bxb1 integrase was used for comparison and showed 0% recombination on all 4 tested target sites.

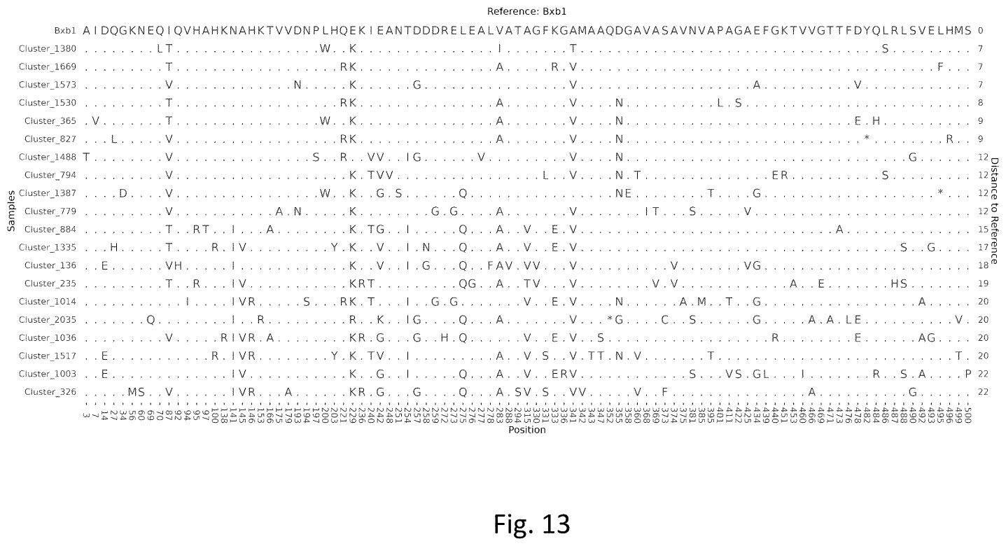

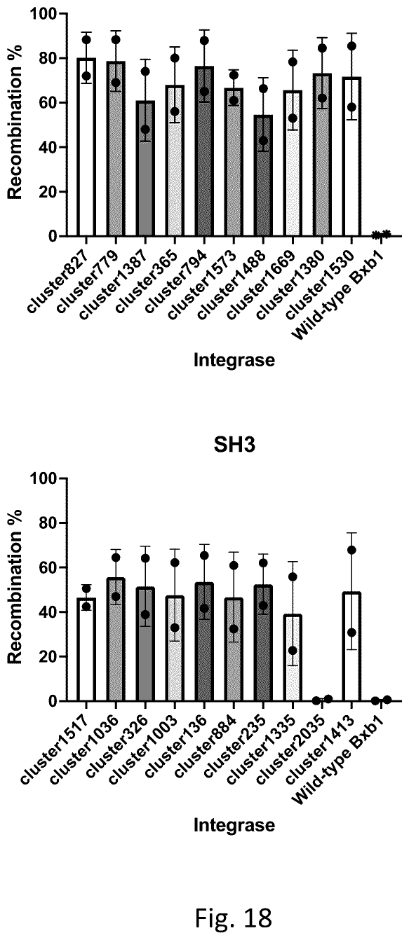

Subsequently, ten clones per library with the highest performance in the Nanopore screen were tested. Sequence alignments for all of them are shown in FIG. 13. Measured recombination efficiencies are presented in FIG. 18.

These experiments show that evolved clones can recombine their new target site in mammalian cells, reaching 89% efficiency, while wild-type Bxb1 is completely inactive on the new target sites.

The pCAGGS-attB-mCherry-attP-EGFP reporter plasmids (FIG. 14) used in the experiments were generated from the pCAGGS-loxP-mCherry-loxP-EGFP plasmid described by Karpinski et al. (2016). The target sites were introduced by PCR with overhang primers and were inserted in the reporter plasmid using NheI and HindIII restriction digestion cloning.

TABLE 7
Primers used for PCR:

| Primer | Primer sequence |
|---|---|
| F | AAAAGCTAGC |
| | (SEQ ID NO: 13116) |
| | -target_site- |
| | CGCCACCATGGTGAGCAA |
| | (SEQ ID NO: 13117) |
| R | AAAAAAGCTTAGATCT |
| | (SEQ ID NO: 13118) |
| | -rev_comp- |
| | CCTGCATCGACCTAGACTAGCTAGAA |
| | (SEQ ID NO: 13119) |

The transient mammalian expression vector EF1a-tagBFP-P2A-Int (FIG. 15) was generated from the EF1a-Rec-P2A-EGFP vector as described by Lansing et al. (2020) and Lansing et al. (2022). The integrases were inserted in the expression plasmid by restriction digest using XbaI and BsrGI.

### Example 6: SH2 Integration Experiment

This experiment shows the successful integration of a donor nucleic acid sequence into the SH2 locus using an evolved LSR.

A pTwist-CMV plasmid (Twist Bioscience) was used for expression of the SH2 clone 20 integrase (SEQ ID NO: 12437) with the Nucleoplasmin NLS on the C-terminus of the recombinase. The SH2-c120-NP was cloned via HindIII and NheI restriction sites into the pTwist-CMV expression vector. The donor plasmid pTwist-Kan-DNR3-P52 derived from a pTwist-Kan high copy cloning vector (Twist Bioscience) containing an SH2-P52 target site (5′-GTGGTTTGTCTGGTCAACCACCGCGGTCTCAGTGGTGTACGGTACAAACCCA-3′; SEQ ID NO: 13126) and a turboGFP followed by SV40 poly(A) sequence under a short CMV promotor (without enhancer and chimeric intron sequences). The fragment attP52-CMV-turboGFP-SV40poly(A) was cloned into the pTwist-Kan high copy cloning vector (Twist Bioscience).

Expi293F cells (Thermo Fisher Scientific) were cultured in DMEM (Gibco) with 10% FBS (Gibco) and 1% penicillin-streptomycin (10,000 U/ml, Thermo Fisher Scientific) at 37° C. and 5% CO₂. Trypsin-EDTA (Gibco) was used for dissociation of the cells for splitting.

For the integration assay, 30,000 Expi293F cells per well were seeded in a 96-well plate. The next day, 25 ng of the pTwist-CMV-SH2c120-NP expression plasmid and 200 ng of the pDNR3-attP52 donor plasmid were transfected using Lipofectamine 2000 Transfection Reagent (Thermo Fisher Scientific). The cells were analyzed with a MACSQuant X (Miltenyi Biotec) 72 h after transfection. The cells were gated for single cells and then for transfected population (GFP⁺ cells). The transfection efficiency of the sample transfected with the recombinase and donor plasmids was 49.6%.

72 h post-transfection, the cells were harvested and gDNA was extracted using the Fast extract DNA solution (VWR) following the manufacturer's instructions.

1 μl of the extracted gDNA (untreated cells were used as a control) was used for junction PCR with MyTaq polymerase (Bioline) in 25 μl PCR reaction. The junction PCR allows to check for correct donor integration in the SH2-B target site in the genome by amplifying the DNA fragment between the genomic locus and the integrated DNA sequence. PCR was performed using a primer specific for the sequence to the genomic sequence upstream (5′) or downstream (3′) of the SH2-B target site, and another primer specific to the sequence that was integrated by the recombinase (the donor plasmid). Primers used for the left junction (5′) PCR: GCAATGAGGTCCCAGATCCT (SEQ ID NO: 13120, binds in the genomic sequence, coordinates of the primer binding site in the human genome next to the SH2-B target site: chr13:74,941,518-74,941,537) and TGGCGGTCATATTGGACATGA (SEQ ID NO: 13121, binds in the donor plasmid downstream the target site); expected product size: 435 bp. Primers used for the right junction (3′) PCR: TCGGGCTTCCCATACAATCG (SEQ ID NO: 13122, binds in the donor plasmid upstream the target site) and CTTGCGCCTCCAGAAACATTG (SEQ ID NO: 13123, binds in the genomic sequence, coordinates of the primer binding site in the human genome next to the SH2-B target site: chr13:74,941,098-74,941,118); expected product size: 618 bp. Cycling program: 95° C.—5 min, (95° C.—15s, 56° C.—15s, 72° C.—15s)×35, 72° C.—3 min.

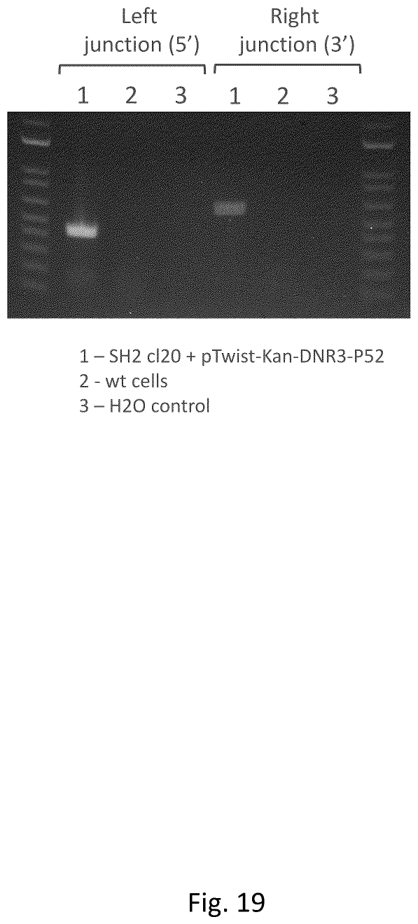

5 μl of the PCR product was run on a 22 well 1% Agarose E-Gel (Thermo Fisher Scientific) for 20 min. E-Gel 1 Kb Plus Express DNA Ladder was used to analyze the PCR product size. Gel photo (FIG. 19) was taken with the E-Gel Power Snap Plus Electrophoresis System (Thermo Fisher Scientific). On the gel picture, two PCR bands are present in the sample lanes, where the gDNA from the cells transfected with SH2 c120 recombinase and pTwist-Kan-DNR3-P52 donor was used as a template. The size of the bands matches the expected sizes of 435 bp for the left junction (5′), and 618 bp for the right junction (3′), indicating successful integration of the donor plasmid into the SH2 genomic locus. No PCR product was amplified in the samples where gDNA of untreated cells (wt cells) was used as a template, indicating specificity of the PCR assay (see control lanes 2 in FIG. 19; control lanes 3 show no amplicons for the H₂O negative controls in the PCR reaction mixture).

1 μl of the PCR product was sent for Sanger Sequencing to Microsynth AG with the primer GCAATGAGGTCCCAGATCCT (SEQ ID NO: 13124) for the left junction PCR and the primer TCGGGCTTCCCATACAATCG (SEQ ID NO: 13125) for the right junction PCR. Sequencing results confirmed successful integration of the donor nucleic acid sequence into the SH2 locus.

### CITED NON-PATENT LITERATURE

Anzalone A V, Gao X D, Podracky C J, Nelson A T, Koblan L W, Raguram A, Levy J M, Mercer JAM, Liu D R. Programmable deletion, replacement, integration and inversion of large DNA sequences with twin prime editing. Nat Biotechnol. 2022 May; 40(5):731-740.
Aznauryan E, Yermanos A, Kinzina E, Devaux A, Kapetanovic E, Milanova D, Church G M, Reddy S T. Discovery and validation of human genomic safe harbour sites for gene and cell therapies. Cell Reports Methods 2022 January: 2(1):100154.
Bai H, Sun M, Ghosh P, Hatfull G F, Grindley N D, Marko J F. Single-molecule analysis reveals the molecular bearing mechanism of DNA strand exchange by a serine recombinase. Proc Natl Acad Sci USA. 2011 May 3; 108(18):7419-24. doi: 10.1073/pnas.1018436108. Epub 2011 Apr. 18. PMID: 21502527; PMCID: PMC3088605.
Buchholz F., Hauber J. (2011). In vitro evolution and analysis of HIV-1 LTR-specific recombinases. Methods 53, 102-109.
Buchholz, F., Stewart, A. F. (2001). Alteration of Cre recombinase site specificity by substrate-linked protein evolution. Nat Biotechnol 19, 1047-1052.
Durrant M G, Fanton A, Tycko J, Hinks M, Chandrasekaran S S, Perry N T, Schaepe J, Du P P, Lotfy P, Bassik M C, Bintu L, Bhatt A S, Hsu P D. Systematic discovery of recombinases for efficient integration of large DNA sequences into the human genome. Nat Biotechnol. 2023 April; 41(4):488-499. doi: 10.1038/s41587-022-01494-w. Epub 2022 Oct. 10. PMID: 36217031; PMCID: PMC10083194.
Engler C, Kandzia R, Marillonnet S. A one pot, one step, precision cloning method with high throughput capability. PLoS One. 2008; 3(11):e3647. doi: 10.1371/journal.pone.0003647. Epub 2008 Nov. 5. PMID: 18985154; PMCID: PMC2574415.
Ghosh P, Pannunzio N R, Hatfull G F. Synapsis in phage Bxb1 integration: selection mechanism for the correct pair of recombination sites. J Mol Biol. 2005 Jun. 3; 349(2):331-48. doi: 10.1016/j.jmb.2005.03.043. Epub 2005 Apr. 7. PMID: 15890199.
Groth A C, Fish M, Nusse R, Calos M P. Construction of transgenic Drosophila by using the site-specific integrase from phage phiC31. Genetics. 2004 April; 166(4):1775-82. doi: 10.1534/genetics.166.4.1775. PMID: 15126397; PMCID: PMC1470814.
Henikoff S and Henikoff J G. 1989. Amino acid substitution matrices from protein blocks. Proc. Natl. Acad. Sci. USA 89(22):10915-10919.
Karpinski J., Hauber I., Chemnitz J., Schäfer C., Paszkowski-Rogacz M., Chakraborty D., Beschorner N., Hofmann-Sieber H., Lange U. C., Grundhoff A. (2016). Directed evolution of a recombinase that excises the provirus of most HIV-1 primary isolates with high specificity. Nature Biotechnology 34, 401-409.
Karst S. M., Ziels R. M., Kirkegaard R. H., Sørensen E. A., McDonald D., Zhu Q., Knight R., Albertsen M. (2021). High-accuracy long-read amplicon sequences using unique molecular identifiers with nanopore or PacBio sequencing. Nat Methods 18:165-169; available from: https://doi.org/10.1038%2Fs41592-020-01041-y.
Keravala A, Groth A C, Jarrahian S, Thyagarajan B, Hoyt J J, Kirby P J, Calos M P. A diversity of serine phage integrases mediate site-specific recombination in mammalian cells. Mol Genet Genomics. 2006 August; 276(2):135-46. doi: 10.1007/s00438-006-0129-5. Epub 2006 May 13. PMID: 16699779.
Kim A I, Ghosh P, Aaron M A, Bibb L A, Jain S, Hatfull G F. Mycobacteriophage Bxb1 integrates into the Mycobacterium smegmatis groEL1 gene. Mol Microbiol. 2003 October; 50(2):463-73. doi: 10.1046/j.1365-2958.2003.03723.x. PMID: 14617171.
Lansing F, Paszkowski-Rogacz M, Schmitt L T, Schneider P M, Rojo Romanos T, Sonntag J, Buchholz F. A heterodimer of evolved designer-recombinases precisely excises a human genomic DNA locus. Nucleic Acids Res. 2020 Jan. 10; 48(1):472-485. doi: 10.1093/nar/gkz1078. PMID: 31745551; PMCID: PMC7107906.
Lansing F, Mukhametzyanova L, Rojo-Romanos T, Iwasawa K, Kimura M, Paszkowski-Rogacz M, Karpinski J, Grass T, Sonntag J, Schneider P M, Günes C, Hoersten J, Schmitt L T, Rodriguez-Muela N, Knöfler R, Takebe T, Buchholz F. Correction of a Factor VIII genomic inversion with designer-recombinases. Nat Commun. 2022 Jan. 20; 13(1):422. doi: 10.1038/s41467-022-28080-7. PMID: 35058465; PMCID: PMC8776779.
Low B E, Hosur V, Lesbirel S, Wiles M V. Efficient targeted transgenesis of large donor DNA into multiple mouse genetic backgrounds using bacteriophage Bxb1 integrase. Sci Rep. 2022 Mar. 31; 12(1):5424. doi: 10.1038/s41598-022-09445-w. PMID: 35361849; PMCID: PMC8971409.
Needleman S B, Wunsch C D. A general method applicable to the search for similarities in the amino acid sequence of two proteins. JMB 1970 Vol.: 48(3):443-453.
Olivares E C, Hollis R P, Calos M P. Phage R4 integrase mediates site-specific integration in human cells. Gene. 2001 Oct. 31; 278(1-2):167-76. doi: 10.1016/s0378-1119(01)00711-9. PMID: 11707334.
Pearson W R and Lipman D J. 1988. Proc. Natl. Acad. Sci. USA: 2444-2448.
Pellenz S, Phelps M, Tang W, Hovde B T, Sinit R B, Fu W, Li H, Chen E, Monnat R J jr. New Human Chromosomal Sites with “Safe Harbor” Potential for Targeted Transgene Insertion. Hum Gene Ther. 2019 Jul. 1; 30(7): 814-828.
Rognes T, Flouri T, Nichols B, Quince C, Mahé, F. VSEARCH: a versatile open source tool for metagenomics. PeerJ 2016, 1-22.
Russell J P, Chang D W, Tretiakova A, Padidam M. Phage Bxb1 integrase mediates highly efficient site-specific recombination in mammalian cells. Biotechniques. 2006 April; 40(4):460, 462, 464. doi: 10.2144/000112150. PMID: 16629393.

Rutherford K, Yuan P, Perry K, Sharp R, Van Duyne G D. Attachment site recognition and regulation of directionality by the serine integrases. Nucleic Acids Res. 2013 September; 41(17):8341-56. doi: 10.1093/nar/gkt580. Epub 2013 Jul. 2. PMID: 23821671; PMCID: PMC3783163.
Smith M C M. Phage-encoded Serine Integrases and Other Large Serine Recombinases. Microbiol Spectr. 2015 August; 3(4). doi: 10.1128/microbiolspec.MDNA3-0059-2014. PMID: 26350324.
Thomason L C, Calendar R, Ow D W. Gene insertion and replacement in Schizosaccharomyces pombe mediated by the Streptomyces bacteriophage phiC31 site-specific recombination system. Mol Genet Genomics. 2001 August; 265(6):1031-8. doi: 10.1007/s004380100498. PMID: 11523775.
Thorpe H M, Smith M C. In vitro site-specific integration of bacteriophage DNA catalyzed by a recombinase of the resolvase/invertase family. Proc Natl Acad Sci USA. 1998 May 12; 95(10):5505-10. doi: 10.1073/pnas.95.10.5505.
Thorpe H M, Wilson S E, Smith M C. Control of directionality in the site-specific recombination system of the Streptomyces phage phiC31. Mol Microbiol. 2000 October; 38(2):232-41. doi: 10.1046/j.1365-2958.2000.02142.x. PMID: 11069650.
Thyagarajan B, Olivares E C, Hollis R P, Ginsburg D S, Calos M P. Site-specific genomic integration in mammalian cells mediated by phage phiC31 integrase. Mol Cell Biol. 2001 June; 21(12):3926-34. doi: 10.1128/MCB.21.12.3926-3934.2001. PMID: 11359900; PMCID: PMC87055.
Van Duyne G D, Rutherford K. Large serine recombinase domain structure and attachment site binding. Crit Rev Biochem Mol Biol. 2013 September-October; 48(5):476-491. doi: 10.3109/10409238.2013.831807. Epub 2013 Aug. 28. PMID: 23980849.
Xu Z, Lee N C, Dafhnis-Calas F, Malla S, Smith M C, Brown W R. Site-specific recombination in Schizosaccharomyces pombe and systematic assembly of a 400 kb transgene array in mammalian cells using the integrase of Streptomyces phage phiBT1. Nucleic Acids Res. 2008 January; 36(1):e9. doi: 10.1093/nar/gkm1123. Epub 2007 Dec. 20. PMID: 18096621; PMCID: PMC2248738.
Yamaguchi S, Kazuki Y, Nakayama Y, Nanba E, Oshimura M, Ohbayashi T. A method for producing transgenic cells using a multi-integrase system on a human artificial chromosome vector. PLoS One. 2011 Feb. 24; 6(2):e17267. doi: 10.1371/journal.pone.0017267. PMID: 21390305; PMCID: PMC3044732.
Yarnall M T N, Ioannidi E I, Schmitt-Ulms C, Krajeski R N, Lim J, Villiger L, Zhou W, Jiang K, Garushyants S K, Roberts N, Zhang L, Vakulskas C A, Walker J A 2nd, Kadina A P, Zepeda A E, Holden K, Ma H, Xie J, Gao G, Foquet L, Bial G, Donnelly S K, Miyata Y, Radiloff D R, Henderson J M, Ujita A, Abudayyeh O O, Gootenberg J S. Drag-and-drop genome insertion of large sequences without double-strand DNA cleavage using CRISPR-directed integrases. Nat Biotechnol. 2023 April; 41(4):500-512. doi: 10.1038/s41587-022-01527-4. Epub 2022 Nov. 24. PMID: 36424489; PMCID: PMC10257351.
Zhu F, Gamboa M, Farruggio A P, Hippenmeyer S, Tasic B, Schüle B, Chen-Tsai Y, Calos M P. DICE, an efficient system for iterative genomic editing in human pluripotent stem cells. Nucleic Acids Res. 2014 March; 42(5):e34. doi: 10.1093/nar/gkt1290. Epub 2013 Dec. 4. PMID: 24304893; PMCID: PMC3950688.
Zurek P J., Knyphausen P., Neufeld K., Pushpanath A., Hollfelder F. (2020). UMI-linked consensus sequencing enables phylogenetic analysis of directed evolution. Nature Communications 11; available from: https://doi.org/10.1038/s41467-020-19687-9.

1. Method for generating a target-specific large serine recombinase (LSR), the method comprising the steps of:
a) generating a plurality of first variants of an LSR by introducing one or more mutations into a sequence encoding the LSR;
b) generating a library of expression vectors, wherein each expression vector comprises a first region comprising the sequence encoding one of the first variants of the LSR from among the plurality, and a second region comprising at least two first target sites of the LSR, wherein the two first target sites are different from each other;
c) introducing the library of expression vectors into host cells;
d) culturing the host cells and expressing the first variant LSR;
e) isolating plasmid DNA from the culture of host cells;
f) determining whether a portion of the second region on the expression vector has been excised between the two first target sites by the first variant LSR;
g) generating a plurality of second LSR variants by introducing one or more mutations into the sequence encoding those first LSR variants that excised the portion of the second region on the expression vector between the two first target sites;
h) repeating steps b) to f) optionally with at least one second target site, wherein the at least one second target site differs from the first target sites in at least one nucleotide.

2. The method according to claim 1, further comprising the step of repeating steps g) and h), preferably wherein steps g) and h) are repeated at least twice to at least 30 times.

3. The method according to claim 1 or 2, wherein the LSR in step a) as basis for the plurality of first variant LSRs is a naturally occurring LSR.

4. The method according to any one of claims 1 to 3, wherein the two target sites in the second region of the expression vector are spaced apart by at least 50 nucleotides.

5. The method according to any one of claims 1 to 4, wherein the determining step f) comprises:
i) sequencing of the first and the second region of the expression vector; or ii) performing restriction digestion on the sequence of the expression vector comprising the two target sites followed by analysis of the digestion fragments.

6. The method according to any one of claims 1 to 5, further comprising the step of removing inactive variants of the variant LSR from the library of expression vectors.

7. A variant large serine recombinase (LSR) obtainable by the method according to any of claims 1 to 6, wherein the amino acid sequence of the variant LSR differs in at least one amino acid from the amino acid sequence of the LSR from which the variant is generated in step a), and from the amino acid sequence of any other naturally occurring serine recombinase, preferably wherein the variant comprises or consists of an amino acid sequence having at least 85% identity to one of SEQ ID NOs: 14, 17 to 35, 38 to 56, 60 to 8391, 8395, 8398 to 8403, 8406 to 8412, 8415 to 8422, 8425 to 8429, and 8430 to 13115.

8. The variant LSR according to claim 7, wherein the variant LSR is a variant of an LSR selected from the group consisting of A118, TP901, φRV1, Bxb1, C31, R4, Wβ, Tnpx, Cp36, Dn29, Kp03, Nm60, Pa01, Si74.

9. A nucleic acid or group of nucleic acids encoding a variant LSR according to claim 7 or 8.

10. An expression vector comprising a nucleic acid or group of nucleic acids according to claim 9.

11. A system for integrating a donor DNA into a target nucleic acid, the system comprising
a polypeptide comprising a variant LSR according to claim 7 or 8, or a nucleic acid encoding the same, and
a donor nucleic acid to be inserted into the target nucleic acid.

12. A pharmaceutical composition comprising the variant LSR of claim 7 or 8, the nucleic acid or group of nucleic acids according to claim 9, or the expression vector according to claim 10, and optionally a pharmaceutically acceptable carrier.

13. Use of the variant LSR of claim 7 or 8, the nucleic acid or group of nucleic acids of claim 9, the expression vector of claim 10, the system of claim 11, or the pharmaceutical composition of claim 12, for integrating a nucleic acid sequence of interest into the genome of a subject or cell, wherein the cell does not include cells of the human germ line.

14. The variant LSR according to claim 7 or 8, the nucleic acid or group of nucleic acids of claim 9, the expression vector of claim 10, or the pharmaceutical composition of claim 12 for use in medicine.

15. The variant LSR, the nucleic acid or group of nucleic acids, the expression vector, or the pharmaceutical composition for use of claim 14, for use in the treatment of a genetic disease or disorder, preferably wherein the genetic disease or disorder is a monogenetic disease or disorder.
